(19) United States
(12) Patent Application Publication
(10) Pub. No.: US 2021/0079384 A1
(43) Pub. Date: Mar. 18, 2021

(54) NON-INVASIVE DETECTION OF RESPONSE TO A TARGETED THERAPY

(71) Applicant: The Johns Hopkins University, Baltimore, MD (US)

(72) Inventors: Victor E. Velculescu, Dayton, MD (US); Jillian A. Phallen, Baltimore, MD (US); Alessandro Leal, Baltimore, MD (US); Hatim Husain, Baltimore, MD (US)

(21) Appl. No.: 17/047,013

(22) PCT Filed: Apr. 12, 2019
(86) PCT No.: PCT/US2019/027207

§ 371 Date: Oct. 12, 2020

Related U.S. Application Data
(60) Provisional application No. 62/657,618, filed on Apr. 13, 2018.

(51) Int. Cl. C12N 15/10

(52) U.S. Cl. CPC C12N 15/1072; C12N 15/1065

(57) ABSTRACT

Provided herein are method of determining the efficacy of targeted therapy in a subject by detecting changes in levels of cell-free tumor load (cfTL). In some aspects, the efficacy of targeted therapy is determined a very short time after the targeted therapy is administered. Also provided herein are method of determining resistance to a targeted therapy in a subject by detecting changes in levels of cell-free tumor load (cfTL).

### STATEMENT AS TO FEDERALLY SPONSORED RESEARCH

This invention was made with government support under grant numbers NIH R01 grant (CA121113) awarded by the National Institutes of Health. The government has certain rights in the invention.

### TECHNICAL FIELD

The present disclosure relates generally to the field of cancer. More specifically, this disclosure relates to non-invasive in vitro methods for determining the efficacy of a targeted therapy (e.g., a kinase inhibitor (KI)).

### BACKGROUND

The management of oncogene-addicted cancer has been improved by the development of targeted therapies that act against a variety of cancer dependencies. However, therapeutic efficacy of targeted therapies has been limited by incomplete pharmacological suppression of tumors or through the selection of resistance mutations in sub-clonal populations of tumor cells. Disease monitoring using computed tomography (CT) imaging is the current clinical practice for assessing response to targeted therapy, yet this approach does not fully represent the molecular and pathologic changes occurring in tumors during therapy.

### SUMMARY

With the advent of precision oncology approaches, there is an urgent need to develop improved methods for rapidly detecting responses to targeted therapies. An ultrasensitive measure of cell-free tumor burden was developed using targeted and whole genome sequencing approaches to assess responses to tyrosine kinase inhibitors in advanced lung cancer patients. Patients had a bimodal distribution of cell-free circulating tumor DNA (ctDNA) one to three weeks after therapy, with responders having nearly complete elimination of ctDNA while non-responders had limited changes in ctDNA. Changes in sequence alterations in ctDNA were detectable within hours after treatment in responders. Patients with ctDNA responses had improved progression-free survival (10.8 vs 2.0 months, P<0.001), which was detected on average 40 days earlier and was as predictive as CT imaging. These analyses provide a rapid approach for evaluating therapeutic outcomes to targeted therapies and have important implications for the management of cancer patients and development of new therapeutics.

In one aspect, provided herein are methods of predicting the efficacy of a targeted therapy (e.g., a kinase inhibitor (e.g., a tyrosine kinase inhibitor (TKI))) in a subject having been previously diagnosed with cancer and having received at least one dose of a targeted therapy (e.g., a kinase inhibitor (e.g., a tyrosine kinase inhibitor (TKI))).

In some embodiments, provided herein are methods of determining the efficacy of a targeted therapy in a subject having cancer that include: detecting a first cell-free tumor load (cfTL) in a biological sample isolated from the subject at a first time point, detecting a second cfTL in a biological sample obtained from the subject at a second time point, wherein the subject has received at least one dose of the targeted therapy between the first time point and the second time point, and identifying the targeted therapy as being effective in the subject when the subject exhibits a second cfTL that is reduced as compared to the first cfTL.

In some embodiments of detecting a first and second cfTL (e.g. a first and second cfTL at different time points), detecting the first cfTL includes detecting a first level of the at least one genetic alteration in circulating tumor DNA (ctDNA) in the biological sample isolated from the subject at the first time point, wherein the first cfTL corresponds to the first level of the at least one genetic alteration, and detecting the second cfTL includes detecting a second level of the at least one genetic alteration in circulating tumor DNA (ctDNA) in the biological sample isolated from the subject at the second time point, wherein the second cfTL corresponds to the second level of the at least one genetic alteration. In some embodiments, detecting the first level of the at least one genetic alteration, detecting the second level of the at least one genetic alteration, or both includes using a method selected from the group consisting of: a targeted capture method, a next-generation sequencing method, an array-based method, and combinations thereof. In some embodiments, detecting the first level of the at least one genetic alteration, detecting the second level of the at least one genetic alteration, or both includes: extracting cell-free DNA from blood; ligating a low complexity pool of dual index barcode adapters to the cell-free DNA to generate a plurality of barcode adapter-ligated cell-free DNA segments, capturing the plurality of barcode adapter-ligated cell-free DNA segments, sequencing the plurality of captured barcode adapter-ligated cell-free DNA segments, aligning the sequenced plurality of captured barcode adapter-ligated cell-free DNA segments to a reference genome, and identifying sequence alterations using aligned sequences of multiple distinct molecules containing identical redundant changes.

In some embodiments of any of the methods provided herein that includes detecting at least one genetic alteration, the at least one genetic is a mutation is in an EGFR gene, an ERBB2 gene, or both. In some embodiments of any of the methods provided herein that includes detecting at least one genetic alteration, the at least one genetic alteration is a T790M mutation in the EGFR gene.

In some embodiments of any of the methods provided herein that includes detecting a first and second level of at least one genetic alteration, the second level of the at least one genetic alteration of ctDNA is at least about 90% lower than the first level of the at least one genetic alteration of ctDNA.

In some embodiments of detecting a first and second cfTL (e.g. a first and second cfTL at different time points), detecting the first cfTL comprises detecting a first level of aneuploidy in the biological sample isolated from the subject at the first time point, wherein the first cfTL corresponds to the first level of aneuploidy, and detecting the second cfTL comprises detecting a second level of aneuploidy in the biological sample isolated from the subject at the second time point, wherein the second cfTL corresponds to the second level of aneuploidy. In some embodiments, detecting the first level of aneuploidy, detecting the second level of aneuploidy, or both includes: performing digital karyotyping, next generation sequencing, array-based methods, and combinations thereof. In some embodiments, the method further includes: a) extracting a first sample of cell-free DNA from blood at the first time point, ligating a low complexity pool of dual index barcode adapters to the first cell-free DNA sample to generate a first plurality of barcode adapter-ligated cell-free DNA segments, capturing the first plurality of barcode adapter-ligated cell-free DNA segments, eluting the non-captured cell-free DNA to generate a first non-captured cell-free DNA sample, detecting the first level of aneuploidy in the first non-captured non-capture cell-free DNA; and b) extracting a second sample of cell-free DNA from blood at the second time point, ligating a low complexity pool of dual index barcode adapters to the cell-free DNA to generate a plurality of barcode adapter-ligated cell-free DNA segments, capturing the plurality of barcode adapter-ligated cell-free DNA segments, eluting the non-captured cell-free DNA to generate a second non-captured cell-free DNA sample, detecting the second level of aneuploidy in the second non-captured non-capture cell-free DNA.

In some embodiments of any of the methods provided herein in which a first and second level of circulating tumor DNA (ctDNA) is detected, a second level of at least one genetic alteration in circulating tumor DNA (ctDNA) in the biological sample isolated from the subject at the first time point is not substantially different than a first level of the at least one genetic alteration in circulating tumor DNA (ctDNA) in the biological sample isolated from the subject at the first time point.

In some embodiments of any of the methods provided herein, a biological sample obtained from the subject at the first time point, the second time point, or both comprises blood, plasma, serum, urine, cerebrospinal fluid, saliva, sputum, broncho-alveolar lavage, bile, lymphatic fluid, cyst fluid, stool, uterine lavage, vaginal fluids, ascites, and combinations thereof.

In some embodiments of any of the methods provided herein, the targeted therapy is a kinase inhibitor. In some embodiments, the kinase inhibitor is a tyrosine kinase inhibitor. In some embodiments, the kinase inhibitor is selected from the group consisting of: afatinib, crizotinib, erlotinib, gefitinib, osimertinib, and combinations thereof.

In some embodiments of any of the methods provided herein, the subject has been previously administered a different treatment or targeted therapy and the different treatment or targeted therapy was determined not to be therapeutically effective. In some embodiments of any of the methods provided herein, the method further includes administering one or more additional doses of the targeted therapy identified as being effective to the subject.

In some embodiments of any of the methods provided herein, the method further includes administering a therapeutic intervention to the subject. In some embodiments, the therapeutic intervention is selected from the group consisting of: a different targeted therapy, an antibody, an adoptive T cell therapy, a chimeric antigen receptor (CAR) T cell therapy, an antibody-drug conjugate, a cytokine therapy, a cancer vaccine, a checkpoint inhibitor, radiation therapy, surgery, a chemotherapeutic agent, and combinations thereof.

In some embodiments of any of the methods provided herein, a subject has a cancer selected from the group consisting of: a head and neck cancer, a central nervous system cancer, a lung cancer, a mesothelioma, an esophageal cancer, a gastric cancer, a gall bladder cancer, a liver cancer, a pancreatic cancer, a melanoma, an ovarian cancer, a small intestine cancer, a colorectal cancer, a breast cancer, a sarcoma, a kidney cancer, a bladder cancer, a uterine cancer, a cervical cancer, and a prostate cancer. In some embodiments, the cancer is a lung cancer, and the lung cancer is non-small cell lung cancer. In some embodiments, the cancer comprises a population of cancer cells that harbor an EGFR mutation, a ERBB2 mutation, or both.

In some embodiments of any of the methods provided herein in which cfTL is determined a first and second time point, the second time point is between about 1 week to about 4 weeks after the first time point. In some embodiments, the second time point is about 16 days after the first time point. In some embodiments, the second time point is about 6 days after the first time point.

In some embodiments, provided herein are methods of determining response to a targeted therapy in a subject having cancer that include: detecting a first level of at least one genetic alteration in circulating tumor DNA (ctDNA) in a biological sample isolated from the subject at a first time point, detecting a second level of the at least one genetic alteration in circulating tumor DNA (ctDNA) in a biological sample obtained from the subject at a second time point, wherein the subject has received at least one dose of the targeted therapy between the first time point and the second time point, and identifying the subject as responding to the targeted therapy when the second level of the at least one genetic alteration is substantially increased as compared to the first level of the at least one genetic alteration. In some embodiments, the second time point is about 4 to about 12 hours after the first time point. In some embodiments, the second time point is about 4 to about 12 hours after the first time point.

In some embodiments, provided herein are methods of determining poor efficacy of a targeted therapy in a subject having cancer that include: detecting a first cell-free tumor load (cfTL) in a biological sample isolated from the subject at a first time point, detecting a second cfTL in a biological sample obtained from the subject at a second time point, wherein the subject has received at least one dose of the targeted therapy between the first time point and the second time point, and identifying the targeted therapy as having poor efficacy in the subject when the subject exhibits a second cfTL that is not substantially reduced as compared to the first cfTL. In some embodiments, the second time point is between about 1 week to about 4 weeks after the first time point. In some embodiments, the subject is identified as having poor prognosis when the targeted therapy was identified as having poor efficacy. In some embodiments, the poor prognosis is selected from the group consisting of: shorter progression-free survival, lower overall survival, and combinations thereof.

In some embodiments of any of the methods of determining poor efficacy of a targeted therapy in a subject having cancer provided herein, the method further includes administering a therapeutic intervention to the subject, wherein the therapeutic intervention is not the targeted therapy. In some embodiments, the therapeutic intervention is selected from: a different targeted therapy, an antibody, an adoptive T cell therapy, a chimeric antigen receptor (CAR) T cell therapy, an antibody-drug conjugate, a cytokine therapy, a cancer vaccine, a checkpoint inhibitor, radiation therapy, surgery, a chemotherapeutic agent, and combinations thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 is a schematic overview of cfTL determination and prediction of therapeutic response. Liquid biopsies from metastatic non-small-cell lung cancer (mNSCLC) patients undergoing treatment with tyrosine kinase inhibition (TKI) were analyzed at baseline and 6-22 days after treatment. The TEC-Seq approach was used to directly identify sequence alterations across 58 genes encompassing 80,930 bases sequenced to >30,000× coverage, and whole-genome approaches were used to identify copy number changes in cfDNA. Cell-free tumor load (cfTL) was determined as the mutant allele fraction of the most abundant alteration in a clone targeted by TKI for patients with detected sequence alterations, or as the presence or absence of aneuploidy based on PA score in patients without detectable sequence alterations. Prediction of therapeutic response to targeted therapy based on ctDNA dynamics was assessed through changes in cfTL from baseline to day 6-22 after treatment whereas response assessment through CT imaging was performed 4-7 weeks after treatment.

FIG. 2A is line graphs showing ctDNA changes for a responder patient (CGPLLU12) treated with osimertinib (left) and a non-responder patient (CGPLLU244) treated with (right). Mutant allele fractions of clones identified in cfDNA through the TEC-Seq approach are shown for each time point analyzed with the ctDNA clone representing cfTL shown in bright green and treatment initiation highlighted with a red arrow. RECIST 1.1 sum of longest diameters (SLD, gray boxes) were measured from CT scans at intervals during therapy.

FIG. 2B is line graphs showing copy number changes identified in cfDNA from analyses of whole-genome data at each time point analyzed as Z scores (burgundy dots) for each chromosome arm and PA scores (orange diamonds) for a responder patient (CGPLLU2) treated with osimertinib (left) and a non-responder patient (CGPLLU244) treated with (right).

FIG. 2C shows computer tomography (CT) images showing representative tumor lesions (circled in red) at different time points for a responder patient (CGPLLU12) treated with osimertinib (left) and a non-responder patient (CGPLLU244) treated with (right) FIG. 3A is a line graph showing changes in cfTL (P=0.002: responders, P=0.625: non-responders, Wilcoxon signed rank test) in radiographic responders (blue) and radiographic non-responders (orange) from baseline to day 6-22 post treatment.

FIG. 3B is a line graph showing PA score (P=0.002: responders, P=0.875: non-responders, Wilcoxon signed rank test) in radiographic responders (blue) and radiographic non-responders (orange) from baseline to day 6-22 post treatment.

FIG. 3C is line graph showing the number of mutations (P=0.006: responders, P=1.000: non-responders, Wilcoxon signed rank test) in radiographic responders (blue) and radiographic non-responders (orange) from baseline to day 6-22 post treatment.

FIG. 4A is a line graph showing changes in the levels of ctDNA for six patients at baseline and at four to twelve hours after the initiation of targeted therapy. Emerging ctDNA alterations are depicted in red.

FIG. 4B is a line graph showing changes in the levels of cfDNA extracted from six patients at baseline and at four to twelve hours after the initiation of targeted therapy. Emerging ctDNA alterations are depicted in red.

FIG. 5A is a bar graph showing changes in cfTL from baseline to days 6-22 post treatment clustering patients with reduction of cfTL≥98% as ctDNA responders and ≤98% as ctDNA non-responders.

FIG. 5B is a graph showing cfTL at days 6-22 post treatment and PFS for patients analyzed with radiographic assessment in the right column denoting partial response (PR), stable disease (SD), unmeasureable disease (*), or progressive disease (PD). cfTL levels at day 6-22 were significantly different between ctDNA responders and non-responders (P<0.01, Wilcoxon signed rank test).

FIG. 5C is a Kaplan-Meier curve showing PFS for ctDNA responders and non-responders (P<0.001, Mantel-Cox log rank test).

FIG. 5D is a line graph showing time to response assessment based on CT scan (orange) and analyses of ctDNA (blue) with mean times to assessment shown in dotted lines (P<0.0001, Wilcoxon signed rank test).

FIG. 6A shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU88). RECIST 1.1 sum of longest diameters (SLD, gray boxes) were measured from CT scans at intervals during therapy (top).

FIG. 6B shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU99). RECIST 1.1 sum of longest diameters (SLD, gray boxes) were measured from CT scans at intervals during therapy (top).

FIG. 6C shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU315). RECIST 1.1 sum of longest diameters (SLD, gray boxes) were measured from CT scans at intervals during therapy (top).

FIG. 6D shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU86). RECIST 1.1 sum of longest diameters (SLD, gray boxes) were measured from CT scans at intervals during therapy (top).

FIG. 6E shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU89). RECIST 1.1 sum of longest diameters (SLD, gray boxes) were measured from CT scans at intervals during therapy (top).

FIG. 6F shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU319). RECIST 1.1 sum of longest diameters (SLD, gray boxes) were measured from CT scans at intervals during therapy (top).

FIG. 6G shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU14). RECIST 1.1 sum of longest diameters (SLD, gray boxes) were measured from CT scans at intervals during therapy (top).

FIG. 6H shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU324). RECIST 1.1 sum of longest diameters (SLD, gray boxes) were measured from CT scans at intervals during therapy (top).

FIG. 6I shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU97). RECIST 1.1 sum of longest diameters (SLD, gray boxes) were measured from CT scans at intervals during therapy (top).

FIG. 6J shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU245). RECIST 1.1 sum of longest diameters (SLD, gray boxes) were measured from CT scans at intervals during therapy (top).

FIG. 6K shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU246). RECIST 1.1 sum of longest diameters (SLD, gray boxes) were measured from CT scans at intervals during therapy (top).

FIG. 6L shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU294). RECIST 1.1 sum of longest diameters (SLD, gray boxes) were measured from CT scans at intervals during therapy (top).

FIG. 6M shows line graphs showing mutant allele fractions of clones identified in cfDNA through the TEC-Seq approach for each time point analyzed with treatment initiation highlighted with a red arrow (top), and copy number changes identified in cfDNA from analyses of whole-genome data at each time point analyzed as Z scores (burgundy dots) for each chromosome arm and PA scores (orange diamonds) (bottom) for patient (CGPLLU18). RECIST 1.1 sum of longest diameters (SLD, gray boxes) were measured from CT scans at intervals during therapy (top).

FIG. 7 is a graph showing concordance between alterations observed with TEC-seq in the plasma and clinical NGS analyses in the tumor tissue or plasma. The presence of each alteration in matched tumor tissue or plasma specimen evaluated with clinical NGS tests are indicated with dark blue and light blue dots respectively whereas nonconcordant mutations are indicated in orange.

FIG. 8 is a plot showing the timeline of ctDNA analyses, CT assessments and treatment response. Interval between cfTL response assessment at day 6-22 (colored circles) and CT scan response (green squares) depicts the lead time between ctDNA and imaging analyses. Interval between treatment start and CT scan progression (red squares) depicts progression-free survival.

FIG. 9 is a graph showing the change in RECIST 1.1 SLD at median times of radiographic assessment for ctDNA responders (blue lines) and non-responders (orange lines) with the window of cfTL assessment shown in the dotted bracket.

FIG. 10 is a plot showing the correlation of cfTL at day 6-22 and percent reduction in RECIST SLD on initial CT scan. Inset bar chart depicts cfTL at day 6-22 for ctDNA responders (blue) and ctDNA non-responders (orange).

FIG. 11 is a Kaplan-Meier curve showing PFS for patients (n=14) with partial response (blue line) or progressive disease (orange line) based on radiographic imaging obtained 5-7 weeks after treatment initiation.

### DETAILED DESCRIPTION

As used herein, the word “a” or “an” before a noun represents one or more of the particular noun. For example, the phrase “an immunotherapy” encompasses “one or more immunotherapies.”

As used herein, the term “about” means approximately, in the region of, roughly, or around. When used in conjunction with a numerical range, the term “about” modifies that range by extending the boundaries above and below the numerical values set forth. In general, the term “about” is used herein to modify a numerical value above and below the stated value by a variance of 10%.

As used herein, the term “subject” means a vertebrate, including any member of the class mammalia, including humans, domestic and farm animals, and zoo, sports or pet animals, such as mouse, rabbit, pig, sheep, goat, cattle, horse (e.g., race horse), and higher primates. In some embodiments, the subject is a human. In some embodiments, the subject is a human harboring a cancer cell. In some embodiments, the subject is a human harboring a cancer cell, but who is not known to harbor the cancer cell.

The term “treat(ment)” is used herein to denote delaying the onset of, inhibiting, alleviating the effects of, or prolonging the life of a patient suffering from, a condition, e.g., cancer.

The terms “effective amount” and “amount effective to treat” as used herein, refer to an amount or concentration of a composition or treatment described herein, e.g., a targeted therapy (e.g., any targeted therapy described herein), utilized for a period of time (including acute or chronic administration and periodic or continuous administration) that is effective within the context of its administration for causing an intended effect or physiological outcome. For example, effective amounts of a targeted therapy (e.g., any targeted therapy described herein) for use in the present disclosure include, for example, amounts that inhibit the growth of cancer, e.g., tumors and/or tumor cells, improve, delay tumor growth, improve survival for a patient suffering from or at risk for cancer, and improve the outcome of other cancer treatments. As another example, effective amounts of a targeted therapy (e.g., any of the targeted therapies described herein) can include amounts that advantageously affect a tumor microenvironment, e.g., the cell-free tumor load (cfTL), the level of at least one genetic alteration of circulating tumor DNA (ctDNA) (e.g., a mutation), and/or the level of aneuploidy in the ctDNA.

The terms “a reduced level” or a “decreased level” refer to a reduction or decrease in the level of a particular substance or particular substances (e.g., cfTL and/or ctDNA) of at least about 2-fold (e.g., at least about 4-fold, at least about 6-fold, at least about 8-fold, at least about 10-fold, at least about 12-fold, at least about 14-fold, at least about 20-fold) as compared to a reference level or value.

In some embodiments, a reduced level is a reduction of or decrease in a second level of a particular substance(s) or a particular parameter(s) (e.g., cfTL and/or ctDNA) of at least about 1% (e.g., at least about 2%, at least about 4%, at least about 6%, at least about 8%, at least about 10%, at least about 12%, at least about 14%, at least about 16%, at least about 18%, at least about 20%, at least about 22%, at least about 24%, at least about 26%, at least about 28%, at least about 30%, at least about 40%, at least about 45%, at least about 50%, at least about 60%, at least about 65%, at least about 70%, at least about 75%, at least about 80%, at least about 85%, at least about 90%, at least about 95%, or at least about 99%) as compared to the first level of the particular substance or particular parameter.

The terms “an increased level” or a “higher level” refer to an increase of at least about 2-fold (e.g., at least about 4-fold, at least about 6-fold, at least about 8-fold, at least about 10-fold, at least about 12-fold, at least about 14-fold, at least about 20-fold, or more) of a particular substance(s) or a particular parameter(s) (e.g., cfTL and/or ctDNA). In some embodiments, an increased level of at least one genetic alteration present in ctDNA (e.g., at least two, at least three, at least four, at least five, at least six, at least seven, at least eight, at least nine, at least ten, at least twelve, at least fifteen, at least twenty, or more) is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30-fold higher as compared to a first or reference level of the genetic alteration present in ctDNA.

In some embodiments, an increased level of at least one genetic alteration present in ctDNA is an increase of at least about 1% (e.g., at least about 2%, at least about 4%, at least about 6%, at least about 8%, at least about 10%, at least about 12%, at least about 14%, at least about 16%, at least about 18%, at least about 20%, at least about 22%, at least about 24%, at least about 26%, at least about 28%, at least about 30%, at least about 40%, at least about 45%, at least about 50%, at least about 60%, at least about 65%, at least about 70%, at least about 75%, at least about 80%, at least about 85%, at least about 90%, at least about 95%, or at least about 99%) of a second level of the at least one genetic alteration as compared to a first or reference level of the at least one genetic alteration.

The terms “not substantially reduced” or “not substantially decreased” refer to clinically insignificant changes (e.g., a reduction or decrease) in the second level of a particular substance(s) or a particular parameter(s) (e.g., cfTL) as compared to the first level of the particular substance or particular parameter. In contrast, the terms “substantially reduced” or “substantially decreased” refer to clinically significant changes in the second level of a particular substance(s) or a particular parameter(s) (e.g., cfTL) as compared to the first level of the particular substance or particular parameter.

In some embodiments, a not substantially reduced second level of a detected particular substance(s) or a particular parameter(s) (e.g., cfTL) is a reduction or decrease of less than about 10% (e.g., less than about 9%, less than about 8%, less than about 7%, less than about 6%, less than about 5%, less than about 4%, less than about 3%, less than about 2%, less than about 1%, less than about 0.5%, less than about 0.25%, less than about 0.2%, less than about 0.1%, less than about 0.05%, less than about 0.01%) as compared to the first detected level of the particular substance or particular parameter.

In some embodiments, a not substantially reduced level of a particular detected substance(s) or a particular detected parameter(s) (e.g., cfTL) is an increase of at least about 0.5% (e.g., at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, at least about 26%, at least about 28%, at least about 30%, at least about 40%, at least about 45%, at least about 50%, at least about 60%, at least about 65%, at least about 70%, at least about 75%, at least about 80%, at least about 85%, at least about 90%, at least about 95%, or at least about 99%) in the second detected level of the substance or parameter as compared to the first detected level of the substance or parameter.

The terms “substantially increased” refers to clinically significant changes (e.g., an increase) in the second level of a particular substance or particular substances (e.g., at least one genetic alteration of ctDNA) as compared to the first level of the particular substance or particular substances. In contrast, the term “not substantially increased” refers to clinically insignificant changes in the second level of a particular substance(s) or a particular parameter(s) (e.g., cfTL) as compared to the first level of the particular substance or particular parameter.

In some embodiments, a not substantially increased second level of a particular substance(s) or particular parameter(s) (e.g., at least one genetic alteration of ctDNA) is an increase in levels of at least about 0.5% (e.g., at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, at least about 26%, at least about 28%, at least about 30%, at least about 40%, at least about 45%, at least about 50%, at least about 60%, at least about 65%, at least about 70%, at least about 75%, at least about 80%, at least about 85%, at least about 90%, at least about 95%, or at least about 99%) as compared to the first level of particular substance or particular parameter.

A “chemotherapeutic agent” refers to a chemical compound useful in the treatment of a cancer. Chemotherapeutic agents include, e.g., “anti-hormonal agents” or “endocrine therapeutics” which act to regulate, reduce, block or inhibit the effects of hormones that can promote the growth of cancer. Additional classes, subclasses and examples of chemotherapeutic agents are known in the art.

The terms “acquired resistance” and “resistance” when used in reference to a targeted therapy refer to a subsequent state of decreased effectiveness of the targeted therapy (e.g., when the targeted therapy was initially effective). As will be appreciated by those of ordinary skill in the art, resistance to targeted therapy can arise in a subject receiving targeted therapy treatment when a tumor cell in the subject develops a mutation or other molecular lesion that render the tumor cell resistant to the targeted therapy. In some embodiments, when a subject develops resistance to a first targeted therapy, a therapeutic intervention can be administered to the subject (e.g., the therapeutic intervention can be different from the first targeted therapy, including but not limited to, a different targeted therapy, an immunotherapy, a chemotherapy, a surgery, or any of the variety of other therapeutic interventions disclosed herein).

Skilled practitioners will appreciate that a subject can be diagnosed, e.g., by a medical professional, e.g., a physician or nurse (or veterinarian, as appropriate for the patient being diagnosed), as suffering from or at risk for a condition described herein, e.g., cancer, using any method known in the art, e.g., by assessing a subject's medical history, performing diagnostic tests, and/or by employing imaging techniques.

Skilled practitioners will also appreciate that treatment need not be administered to a subject by the same individual who diagnosed the subject (or the same individual who prescribed the treatment for the subject). Treatment can be administered (and/or administration can be supervised), e.g., by the diagnosing and/or prescribing individual, and/or any other individual, including the subject her/himself (e.g., where the patient is capable of self-administration).

The management of oncogene-addicted cancer has been improved by the development of targeted therapies that act against a variety of cancer dependencies (1, 2). However, therapeutic efficacy of targeted therapies has been limited by incomplete pharmacological suppression of tumors or through the selection of resistance mutations in sub-clonal populations of tumor cells. Disease monitoring using computed tomography (CT) imaging is the current clinical practice for assessing response to targeted therapy, yet this approach does not fully represent the molecular and pathologic changes occurring in tumors during therapy. Repeat tissue biopsies of accessible cancer lesions have been used to provide insights into therapeutic decision-making but rarely capture the complexity of intra- and inter-tumoral heterogeneity and are invasive procedures with potential complications. Theoretically, the ability to non-invasively track specific clonal populations of tumor cells through time has the potential to rapidly and dynamically inform therapy sequence and combinatorial strategies. However, there are currently no approved or clinically recognized non-invasive molecularly defined strategies to assess early drug responsiveness or adaptive resistance in cancer patients before radiographic progression.

Cell-free circulating tumor DNA (ctDNA) is released from tumor cells into the circulation and has been detected in patients with early and late stage cancers (3-8). A key challenge of liquid biopsy approaches has been developing methods to detect and characterize small fractions of ctDNA in large populations of total cell-free DNA. A variety of studies have focused on changes in ctDNA during the course of therapy, but have largely focused on the analysis of specific or limited number of alterations that may only represent specific sub-clones of the tumor (9-18). More recent studies have used panels of commonly mutated driver genes to allow detection of multiple driver clones, typically at the time of diagnosis (4, 6, 19-21). However, no study has yet assessed the clinical value of a comprehensive genome-wide analysis of ctDNA alterations to evaluate tumor burden at very early time points following commencement of targeted therapy.

In some embodiments, provided herein are ultrasensitive liquid biopsy approaches that can be used to evaluate patients with advanced non-small cell lung cancer (NSCLC) who have tumor responses or progression on kinase inhibitors (e.g., tyrosine kinase inhibitors). Non-limiting examples of tyrosine kinase inhibitors include afatinib, a second-generation inhibitor of the epidermal growth factor receptor (EGFR) and erb-b2 receptor tyrosine kinase 2 (ERBB2) (22, 23) and osimertinib, a third-generation tyrosine kinase inhibitor targeting EGFR with activating and resistance (T790M) mutations (24, 25). In some embodiments, methods provided herein can be used to assay rapid changes and the overall levels in the amounts of ctDNA that can serve as real-time and predictive biomarkers of patient outcome to a targeted cancer therapy.

A dramatic reduction of cell-free tumor load was seen in patients that were clinical responders, at time points that were within 6-22 days after initiation of therapy. Remarkably, one patient was classified with non-measurable disease at baseline and another with stable disease at first imaging evaluation experienced a complete clearance of ctDNA a few days after TKI initiation. These examples reflect the utility of ctDNA for addressing current unmet clinical needs for real time biomarkers of response and evolution of tumor burden. The tiered complementary approach has the benefit of incorporating sequence mutations in cfDNA that have both qualitative and quantitative characteristics in the type and level of detected alterations, while chromosomal changes add quantitative assessment of genome-wide alterations that are typically present in cancer.

Analysis of extremely early time points within hours after initiation of therapy identified the emergence of new tumor-derived mutations in most responding patients. The detection of new mutations and increase in the levels of existing alterations at this time provided insight into rapid release of ctDNA observed during apoptotic cell death. These analyses open the possibility of extremely early detection of response to targeted therapies.

In some embodiments, provided herein is a new paradigm in cancer therapeutics and drug development in which cfTL molecular response criteria may be used to provide insight into clinical endpoints including overall survival and progression-free survival. Given the heterogeneity of metastatic disease, various cfTL approaches described herein have the advantage of measuring overall tumor burden of clonal populations during selective pressure of targeted therapies. For patients without molecular response, liquid or tissue biopsies can provide additional information related to mechanisms of resistance and provide a context to consider other therapeutic strategies. In some embodiments, cfTL monitoring provides an early biomarker for studies of novel targeted therapies both for established and new molecular targets. Without wishing to be bound by theory, it is thought that combining cfTL response information with early pharmacokinetic data will ultimately provide the biologically effective dose needed for an individual's cancer rather than a maximally tolerated dose.

### Methods of Determining Efficacy of a Targeted Therapy

Provided herein are methods of determining the efficacy of a targeted therapy (e.g., any targeted therapy described herein (e.g., a kinase inhibitor)) in a subject that include: detecting a first cell-free tumor load (cfTL) in a biological sample isolated from the subject at a first time point; detecting a second cfTL in a biological sample obtained from the subject at a second time point, wherein the subject has received at least one dose of the targeted therapy between the first time point and the second time point; and identifying the targeted therapy as being effective in the subject when the subject exhibits a second cfTL that is reduced as compared to the first cfTL.

In some embodiments of any of the methods described herein, detecting the first cfTL includes detecting a first level of at least one genetic alteration present in circulating tumor DNA (ctDNA) in the biological sample isolated from the subject at the first time point, wherein the first cfTL corresponds to the first level of the at least one genetic alteration; and detecting the second cfTL comprises detecting a second level of the at least one genetic alteration present in circulating tumor DNA (ctDNA) in the biological sample isolated from the subject at the second time point, wherein the second cfTL corresponds to the second level of the at least one genetic alteration.

In some embodiments of any of the methods described herein, detecting the first cfTL comprises detecting a first level of aneuploidy in the biological sample isolated from the subject at the first time point, wherein the first cfTL corresponds to the first level of aneuploidy; and detecting the second cfTL comprises detecting a second level of aneuploidy in the biological sample isolated from the subject at the second time point, wherein the second cfTL corresponds to the second level of aneuploidy.

In some embodiments, a targeted therapy is determined to be effective when the level of at least one genetic alteration present in circulating tumor DNA (ctDNA) identified at the second time point is decreased by at least about 2-fold, at least about 3-fold, at least about 4-fold, at least about 5-fold, at least about 6-fold, at least about 7-fold, at least about 8-fold, at least about 9-fold, or at least about 10-fold or more compared to the level of the at least one genetic alteration of circulating tumor DNA (ctDNA) identified at the first time point.

In some embodiments, a targeted therapy is determined to be effective when the level of at least one genetic alteration present in circulating tumor DNA (ctDNA) identified at the second time point is at least about 25% (e.g., at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) lower than the level of the at least one genetic alteration of ctDNA identified at the first time point.

In some embodiments, a targeted therapy is determined to be effective when the circulating tumor DNA (ctDNA) is not observed at the second time point.

Alternatively, a targeted therapy is determined to be effective when the level of aneuploidy identified at the second time point is at least about 25% (e.g., at least about 30%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) lower than the first of aneuploidy identified at the first time point.

In some embodiments, a targeted therapy is determined not to be effective (e.g., the targeted therapy has poor efficacy) when the amount of at least one genetic alteration present in circulating tumor DNA (ctDNA) identified at the second time point is not substantially decreased (e.g., is less than about 10%, less than about 9%, less than about 8%, less than about 7%, less than about 6%, less than about 5%, less than about 4%, less than about 3%, less than about 2%, less than about 1%, less than about 0.5%, less than about 0.25%, less than about 0.2%, less than about 0.1%, less than about 0.05%, or less than about 0.01%) as compared to the amount of the at least one genetic alteration present in circulating tumor DNA (ctDNA) identified at the first time point.

In some embodiments, a targeted therapy is determined not to be effective (e.g., the targeted therapy has poor efficacy) when the level of aneuploidy identified at the second time point is not substantially decreased (e.g., is less than about 10%, less than about 9%, less than about 8%, less than about 7%, less than about 6%, less than about 5%, less than about 4%, less than about 3%, less than about 2%, less than about 1%, less than about 0.5%, less than about 0.25%, less than about 0.2%, less than about 0.1%, less than about 0.05%, or less than about 0.01%) as compared to the level of aneuploidy identified at the first time point.

In some embodiments, a targeted therapy is determined not to be effective (e.g., the targeted therapy has poor efficacy) when the cell-free tumor load (cfTL) identified at the second time point is not substantially decreased (e.g., less than about 10%, less than about 9%, less than about 8%, less than about 7%, less than about 6%, less than about 5%, less than about 4%, less than about 3%, less than about 2%, less than about 1%, less than about 0.5%, less than about 0.25%, less than about 0.2%, less than about 0.1%, less than about 0.05%, less than about 0.01%) as compared to the amount of the cfTL identified at the first time point.

In some embodiments, methods provided herein for determining the efficacy of a targeted therapy include detecting the level of at least one genetic alteration of circulating tumor DNA (ctDNA) present in cell-free DNA, where the cell-free DNA is present in an amount less than about 1500 ng, e.g., less than about 1400 ng, less than about 1300 ng, less than about 1200 ng, less than about 1100 ng, less than about 1000 ng, less than about 900 ng, less than about 800 ng, less than about 700 ng, less than about 600 ng, less than about 500 ng, less than about 400 ng, less than about 300 ng, less than about 200 ng, less than about 150 ng, less than about 100 ng, less than about 95 ng, less than about 90 ng, less than about 85 ng, less than about 80 ng, less than about 75 ng, less than about 70 ng, less than about 65 ng, less than about 60 ng, less than about 55 ng, less than about 50 ng, less than about 45 ng, less than about 40 ng, less than about 35 ng, less than about 30 ng, less than about 25 ng, less than about 20 ng, less than about 15 ng, less than about 10 ng, or less than about 5 ng.

In some embodiments, after determining the efficacy of a targeted therapy administered to a subject, the subject can be administered a diagnostic test (e.g., any of the diagnostic tests disclosed herein) and/or monitored (e.g., according to any of the monitoring methods, schedules, etc. disclosed herein). In some embodiments, after determining the efficacy of a targeted therapy administered to a subject, the subject can be selected for further diagnostic testing (e.g., using any of the diagnostic tests disclosed herein) and/or selected for increased monitoring (e.g., according to any of the increased monitoring methods, schedules, etc. disclosed herein). For example, a subject can be administered a targeted therapy, and the targeted therapy is determined to be effective, and the subject can then be administered a diagnostic test and/or selected for further diagnostic testing (e.g., to confirm the effectiveness of the targeted therapy). As another example, a subject can be administered a targeted therapy, where was determined to be effective, and the subject can then be monitored and/or selected for increased monitoring (e.g., to keep watch for the reemergence of the same or another cancer).

In some embodiments, a targeted therapy is determined to be effective in a subject. In such embodiments, the subject may be administered one or more additional doses of the effective targeted therapy during the course of treatment. In some embodiments, when a targeted therapy is determined to be effective in a subject, the subject may be administered one or more additional doses of the effective targeted therapy during the course of treatment without being administered other therapeutic interventions (e.g. other therapeutic interventions to treat the same condition the targeted therapy treats, e.g., cancer). In some embodiments, when a targeted therapy is determined to be effective in a subject, the subject may be administered one or more additional doses of the effective targeted therapy, and may further be administered one or more therapeutic interventions (e.g., any of the therapeutic interventions disclosed herein) during the course of treatment.

In some embodiments, a targeted therapy is determined not to be effective in a subject (e.g., the targeted therapy has poor efficacy). In such embodiments, the subject may be administered a therapeutic intervention (e.g., any of the therapeutic interventions disclosed herein) that is different that the ineffective targeted therapy (e.g., a different class of targeted therapy or a different targeted therapy within the same type of targeted therapy that was determined to be ineffective) during the course of treatment. As non-limiting examples, a subject may be administered a different a targeted therapy, a chemotherapy, immunotherapy, radiation therapy, and/or surgery. Those of ordinary skill in the art will be aware of suitable therapeutic interventions to administer when the targeted therapy is determined not to be effective.

In some aspects, methods provided herein include obtaining from the subject additional sample(s) at additional time point(s) (e.g., at a third time point, a fourth time point, etc.) and determining the efficacy of a targeted therapy at the additional time point(s).

In some aspects, the second time point is about one to about four weeks (e.g., about one to about three weeks, about one to about two weeks, about two to about four weeks, about two to about three weeks, about three weeks to about four weeks; about 2 days to about 30 days, about 2 days to about 28 days, about 2 days to about 26 days, about 2 days to about 24 days, about 2 days to about 22 days, about 2 days to about 20 days, about 2 days to about 18 days, about 2 days to about 16 days, about 2 days to about 14 days, about 2 days to about 12 days, about 2 days to about 10 days, about 2 days to about 8 days, about 2 days to about 6 days, about 2 days to about 4 days, about 4 days to about 30, about 4 days to about 28 days, about 4 days to about 26 days, about 4 days to about 24 days, about 4 days to about 22 days, about 4 days to about 20 days, about 4 days to about 18 days, about 4 days to about 16 days, about 4 days to about 14 days, about 4 days to about 12 days, about 4 days to about 10 days, about 4 days to about 8 days, about 4 days to about 6 days, about 6 days to about 30, about 6 days to about 28 days, about 6 days to about 26 days, about 6 days to about 24 days, about 6 days to about 22 days, about 6 days to about 20 days, about 6 days to about 18 days, about 6 days to about 16 days, about 6 days to about 14 days, about 6 days to about 12 days, about 6 days to about 10 days, about 6 days to about 8 days, about 8 days to about 30, about 8 days to about 28 days, about 8 days to about 26 days, about 8 days to about 24 days, about 8 days to about 22 days, about 8 days to about 20 days, about 8 days to about 18 days, about 8 days to about 16 days, about 8 days to about 14 days, about 8 days to about 12 days, about 8 days to about 10 days, about 10 days to about 30, about 10 days to about 28 days, about 10 days to about 26 days, about 10 days to about 24 days, about 10 days to about 22 days, about 10 days to about 20 days, about 10 days to about 18 days, about 10 days to about 16 days, about 10 days to about 14 days, about 10 days to about 12 days, about 12 days to about 30, about 12 days to about 28 days, about 12 days to about 26 days, about 12 days to about 24 days, about 12 days to about 22 days, about 12 days to about 20 days, about 12 days to about 18 days, about 12 days to about 16 days, about 12 days to about 14 days, about 14 days to about 30, about 14 days to about 28 days, about 14 days to about 26 days, about 14 days to about 24 days, about 14 days to about 22 days, about 14 days to about 20 days, about 14 days to about 18 days, about 14 days to about 16 days, about 16 days to about 30, about 16 days to about 28 days, about 16 days to about 26 days, about 16 days to about 24 days, about 16 days to about 22 days, about 16 days to about 20 days, about 16 days to about 18 days, about 18 days to about 30, about 18 days to about 28 days, about 18 days to about 26 days, about 18 days to about 24 days, about 18 days to about 22 days, about 18 days to about 20 days, about 20 days to about 30, about 20 days to about 28 days, about 20 days to about 26 days, about 20 days to about 24 days, about 20 days to about 22 days, about 22 days to about 30, about 22 days to about 28 days, about 22 days to about 26 days, about 22 days to about 24 days, about 24 days to about 30, about 24 days to about 28 days, about 24 days to about 26 days, about 26 days to about 30, about 26 days to about 28 days, about 26 days to about 30; about 1 day, about 2 days, about 4 days, about 6 days, about 8 days, about 10 days, about 12 days, about 14 days, about 16 days, about 18 days, about 20 days, about 22 days, about 24 days, about 26 days, about 28 days, or about 30 days) after the first time point.

### Determining, Monitoring, and Treating Resistance to a Targeted Therapy

Also provided herein are methods for determining that a subject that has developed resistance to a targeted therapy (e.g., any of targeted therapy disclosed herein or known in the art), methods for monitoring a subject for the development of resistance to a targeted therapy, and methods for treating such subjects with a different therapeutic intervention.

Provided herein are methods of determining acquired resistance to a targeted therapy in a subject having cancer that include: detecting a first cell-free tumor load (cfTL) in a biological sample isolated from the subject at a first time point; detecting a second cfTL in a biological sample obtained from the subject at a second time point, wherein the subject has received at least one dose of the targeted therapy between the first time point and the second time point; and identifying as having acquired resistance when the second cfTL is not substantially reduced as compared to the first cfTL. In some embodiments, the subject determined to have developed resistance to the targeted therapy exhibits a decreased level of at least one genetic alteration of ctDNA at a time point between the first and second time points (e.g., the level of the genetic alteration of ctDNA initially decreases upon administration of the targeted therapy, but then increases when the subject develops resistance).

Provided herein are methods of determining acquired resistance to a targeted therapy in a subject having cancer that include: detecting a first level of at least one genetic alteration in circulating tumor DNA (ctDNA) in a biological sample isolated from the subject at a first time point; detecting a second level of the at least one genetic alteration in circulating tumor DNA (ctDNA) in a biological sample obtained from the subject at a second time point, wherein the subject has received at least one dose of the targeted therapy between the first time point and the second time point; and identifying the subject as having acquired resistance when the second level of the at least one genetic alteration is substantially increased as compared to the first level of the at least one genetic alteration.

Provided herein are methods of determining acquired resistance to a targeted therapy in a subject having cancer that include: detecting a first level of aneuploidy in a biological sample isolated from the subject at a first time point; detecting a second level of aneuploidy in a biological sample obtained from the subject at a second time point, wherein the subject has received at least one dose of the targeted therapy between the first time point and the second time point; and identifying the subject as having acquired resistance when the second level of aneuploidy is substantially increased as compared to the first level of aneuploidy.

In some embodiments, a subject is determined not to have developed resistance to a targeted therapy when the amount of at least one genetic alteration of circulating tumor DNA (ctDNA) identified at the second time point is decreased by at least about 2-fold, at least about 3-fold, at least about 4-fold, at least about 5-fold, at least about 6-fold, at least about 7-fold, at least about 8-fold, at least about 9-fold, at least about 10-fold or more compared to the amount of the at least one genetic alteration of circulating tumor DNA (ctDNA) identified at the first time point. In some embodiments, a subject is determined not to have developed resistance to a targeted therapy when the amount of at least one genetic alteration of circulating tumor DNA (ctDNA) identified at the second time point is decreased by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or more compared to the amount of the at least one genetic alteration of circulating tumor DNA (ctDNA) identified at the first time point. In some embodiments, a subject is determined not to have developed resistance to a targeted therapy when circulating tumor DNA (ctDNA) is not observed at the second time point.

In some embodiments, provided herein are methods of determining that a subject has not developed resistance to a targeted therapy (e.g., any of the targeted therapies disclosed herein or known in the art), including: detecting a first cell-free tumor load (cfTL) in a biological sample isolated from the subject at a first time point; detecting a second cfTL in a biological sample obtained from the subject at a second time point, wherein the subject has received at least one dose of the targeted therapy between the first time point and the second time point; and identifying the subject as having not acquired resistance when the second cfTL is reduced as compared to the first cfTL.

In some embodiments, provided herein are methods of determining that a subject has developed resistance to a targeted therapy (e.g., any of the targeted therapies disclosed herein or known in the art), including: detecting a first cell-free tumor load (cfTL) in a biological sample isolated from the subject at a first time point; detecting a second cfTL in a biological sample obtained from the subject at a second time point, wherein the subject has received at least one dose of the targeted therapy between the first time point and the second time point; and identifying the subject as having acquired resistance when the second cfTL is not substantially reduced as compared to the first cfTL.

In some embodiments, the subject determined to have developed resistance to the targeted therapy exhibits an increased cell-free tumor load (cfTL) at a time point between the first and second time points (e.g., cell-free tumor load (cfTL) initially increases upon administration of the targeted therapy, but then decreases when the subject develops resistance).

In some embodiments, detecting and comparing cfTL levels at different time points results in a more rapid determination of whether the subject has developed resistance than conventional methods (e.g., imaging or scanning).

In some embodiments, a subject is identified as having developed resistance to an administered targeted therapy when the second cfTL not substantially reduced as compared to the first cfTL (e.g., an increase in second cfTL of less than about 10%, less than about 9%, less than about 8%, less than about 7%, less than about 6%, less than about 5%, less than about 4%, less than about 3%, less than about 2%, less than about 1%, less than about 0.5%, less than about 0.25%, less than about 0.2%, less than about 0.1%, less than about 0.05%, or less than about 0.01% as compared to the first cfTL).

In some embodiments, methods of determining that a subject that has developed resistance to a targeted therapy (e.g., any of targeted therapy disclosed herein or known in the art) include using any of the methods disclosed herein for detecting the presence or level of at least one genetic alteration of circulating tumor DNA (ctDNA).

In some embodiments, a subject is determined to have developed resistance to a targeted therapy when that targeted therapy is no longer effective or is less effective than it was when first administered. For example, a subject can be determined to have developed resistance to a targeted therapy when the targeted therapy is at least 20%, 25%, 30%, 35%, 40% 45%, 50%, 55%, 60%, 65%, 70%, 75%, 80%, 85%, 90%, 95%, or 100%, or any percentage within between, less effective than when the targeted therapy was first administered. The effectiveness of a targeted therapy, both when it is first administered and during the course of the treatment, can be determined by any of a variety of methods and techniques. For example, the size and/or position of the tumor (as determined, e.g., by scanning or imaging technologies), the number of cancer cells, the amount of cell-free DNA, and/or the amount of genetic alterations of circulating tumor DNA can be determined and used to assess whether a subject has developed resistance to the targeted therapy. Other suitable methods and techniques are known in the art. In some embodiments, after determining that a subject that has developed resistance to a targeted therapy, a different targeted therapy and/or therapeutic intervention (e.g., any of the therapeutic interventions disclosed herein or known in the art) is selected and/or administered to the subject.

In some embodiments, methods for monitoring a subject for the development of resistance to a targeted therapy (e.g., any of targeted therapy disclosed herein or known in the art) include using any of the methods disclosed herein for detecting the presence or level of at least one genetic alteration of circulating tumor DNA (ctDNA).

In some embodiments, methods for treating a subject that has developed resistance to a therapeutic intervention (e.g., any of the therapeutic interventions disclosed herein or known in the art) include using any of the methods disclosed herein for detecting at least one genetic alteration of circulating tumor DNA.

In some embodiments methods provided herein for determining that a subject that has developed resistance to a targeted therapy, for monitoring a subject for the development of resistance to a targeted therapy, and/or for treating such subjects with a different therapeutic intervention include determining the level of at least one genetic alteration of circulating tumor DNA present in cell-free DNA, where the circulating tumor DNA represents 100% of the cell-free DNA.

In some aspects, methods provided herein include obtaining from the subject additional sample(s) at additional time point(s) (e.g., at a third time point, a fourth time point, etc.) and determining whether a subject has developed resistance to a targeted therapy at the additional time point(s).

In some aspects, the second time point is about one to about four weeks (e.g., about one to about three weeks, about one to about two weeks, about two to about four weeks, about two to about three weeks, about three weeks to about four weeks; about 2 days to about 30 days, about 2 days to about 28 days, about 2 days to about 26 days, about 2 days to about 24 days, about 2 days to about 22 days, about 2 days to about 20 days, about 2 days to about 18 days, about 2 days to about 16 days, about 2 days to about 14 days, about 2 days to about 12 days, about 2 days to about 10 days, about 2 days to about 8 days, about 2 days to about 6 days, about 2 days to about 4 days, about 4 days to about 30, about 4 days to about 28 days, about 4 days to about 26 days, about 4 days to about 24 days, about 4 days to about 22 days, about 4 days to about 20 days, about 4 days to about 18 days, about 4 days to about 16 days, about 4 days to about 14 days, about 4 days to about 12 days, about 4 days to about 10 days, about 4 days to about 8 days, about 4 days to about 6 days, about 6 days to about 30, about 6 days to about 28 days, about 6 days to about 26 days, about 6 days to about 24 days, about 6 days to about 22 days, about 6 days to about 20 days, about 6 days to about 18 days, about 6 days to about 16 days, about 6 days to about 14 days, about 6 days to about 12 days, about 6 days to about 10 days, about 6 days to about 8 days, about 8 days to about 30, about 8 days to about 28 days, about 8 days to about 26 days, about 8 days to about 24 days, about 8 days to about 22 days, about 8 days to about 20 days, about 8 days to about 18 days, about 8 days to about 16 days, about 8 days to about 14 days, about 8 days to about 12 days, about 8 days to about 10 days, about 10 days to about 30, about 10 days to about 28 days, about 10 days to about 26 days, about 10 days to about 24 days, about 10 days to about 22 days, about 10 days to about 20 days, about 10 days to about 18 days, about 10 days to about 16 days, about 10 days to about 14 days, about 10 days to about 12 days, about 12 days to about 30, about 12 days to about 28 days, about 12 days to about 26 days, about 12 days to about 24 days, about 12 days to about 22 days, about 12 days to about 20 days, about 12 days to about 18 days, about 12 days to about 16 days, about 12 days to about 14 days, about 14 days to about 30, about 14 days to about 28 days, about 14 days to about 26 days, about 14 days to about 24 days, about 14 days to about 22 days, about 14 days to about 20 days, about 14 days to about 18 days, about 14 days to about 16 days, about 16 days to about 30, about 16 days to about 28 days, about 16 days to about 26 days, about 16 days to about 24 days, about 16 days to about 22 days, about 16 days to about 20 days, about 16 days to about 18 days, about 18 days to about 30, about 18 days to about 28 days, about 18 days to about 26 days, about 18 days to about 24 days, about 18 days to about 22 days, about 18 days to about 20 days, about 20 days to about 30, about 20 days to about 28 days, about 20 days to about 26 days, about 20 days to about 24 days, about 20 days to about 22 days, about 22 days to about 30, about 22 days to about 28 days, about 22 days to about 26 days, about 22 days to about 24 days, about 24 days to about 30, about 24 days to about 28 days, about 24 days to about 26 days, about 26 days to about 30, about 26 days to about 28 days, about 26 days to about 30; about 1 day, about 2 days, about 4 days, about 6 days, about 8 days, about 10 days, about 12 days, about 14 days, about 16 days, about 18 days, about 20 days, about 22 days, about 24 days, about 26 days, about 28 days, or about 30 days) after the first time point.

In some aspects, the second time point is about 1 hour to about 7 days (e.g., about 1 hour to about 6 days, about 1 hour to about 5 days, about 1 hour to about 4 days, about 1 hour to about 72 hours, about 1 hour to about 66 hours, about 1 hour to about 60 hours, about 1 hour to about 54 hours, about 1 hour to about 48 hours, about 1 hour to about 42 hours, about 1 hour to about 36 hours, about 1 hour to about 30 hours, about 1 hour to about 24 hours, about 1 hour to about 18 hours, about 1 hour to about 12 hours, about 1 hour to about 6 hours, about 1 hour to about 4 hours, about 1 hour to about 2 hours, about 2 hours to about 7 days, about 2 hours to about 6 days, about 2 hours to about 5 days, about 2 hours to about 4 days, about 2 hours to about 72 hours, about 2 hours to about 66 hours, about 2 hours to about 60 hours, about 2 hours to about 54 hours, about 2 hours to about 48 hours, about 2 hours to about 42 hours, about 2 hours to about 36 hours, about 2 hours to about 30 hours, about 2 hours to about 24 hours, about 2 hours to about 18 hours, about 2 hours to about 12 hours, about 2 hours to about 6 hours, about 2 hours to about 4 hours, about 4 hours to about 7 days, about 4 hours to about 6 days, about 4 hours to about 5 days, about 4 hours to about 4 days, about 4 hours to about 72 hours, about 4 hours to about 66 hours, about 4 hours to about 60 hours, about 4 hours to about 54 hours, about 4 hours to about 48 hours, about 4 hours to about 42 hours, about 4 hours to about 36 hours, about 4 hours to about 30 hours, about 4 hours to about 24 hours, about 4 hours to about 18 hours, about 4 hours to about 12 hours, about 4 hours to about 6 hours, about 6 hours to about 7 days, about 6 hours to about 6 days, about 6 hours to about 5 days, about 6 hours to about 4 days, about 6 hours to about 72 hours, about 6 hours to about 66 hours, about 6 hours to about 60 hours, about 6 hours to about 54 hours, about 6 hours to about 48 hours, about 6 hours to about 42 hours, about 6 hours to about 36 hours, about 6 hours to about 30 hours, about 6 hours to about 24 hours, about 6 hours to about 18 hours, about 6 hours to about 12 hours, about 12 hours to about 7 days, about 12 hours to about 6 days, about 12 hours to about 5 days, about 12 hours to about 4 days, about 12 hours to about 72 hours, about 12 hours to about 66 hours, about 12 hours to about 60 hours, about 12 hours to about 54 hours, about 12 hours to about 48 hours, about 12 hours to about 42 hours, about 12 hours to about 36 hours, about 12 hours to about 30 hours, about 12 hours to about 24 hours, about 12 hours to about 18 hours, about 18 hours to about 7 days, about 18 hours to about 6 days, about 18 hours to about 5 days, about 18 hours to about 4 days, about 18 hours to about 72 hours, about 18 hours to about 66 hours, about 18 hours to about 60 hours, about 18 hours to about 54 hours, about 18 hours to about 48 hours, about 18 hours to about 42 hours, about 18 hours to about 36 hours, about 18 hours to about 30 hours, about 18 hours to about 24 hours, about 24 hours to about 7 days, about 24 hours to about 6 days, about 24 hours to about 5 days, about 24 hours to about 4 days, about 24 hours to about 72 hours, about 24 hours to about 66 hours, about 24 hours to about 60 hours, about 24 hours to about 54 hours, about 24 hours to about 48 hours, about 24 hours to about 42 hours, about 24 hours to about 36 hours, about 24 hours to about 30 hours, about 30 hours to about 7 days, about 30 hours to about 6 days, about 30 hours to about 5 days, about 30 hours to about 4 days, about 30 hours to about 72 hours, about 30 hours to about 66 hours, about 30 hours to about 60 hours, about 30 hours to about 54 hours, about 30 hours to about 48 hours, about 30 hours to about 42 hours, about 30 hours to about 36 hours, about 36 hours to about 7 days, about 36 hours to about 6 days, about 36 hours to about 5 days, about 36 hours to about 4 days, about 36 hours to about 72 hours, about 36 hours to about 66 hours, about 36 hours to about 60 hours, about 36 hours to about 54 hours, about 36 hours to about 48 hours, about 36 hours to about 42 hours, about 42 hours to about 7 days, about 42 hours to about 6 days, about 42 hours to about 5 days, about 42 hours to about 4 days, about 42 hours to about 72 hours, about 42 hours to about 66 hours, about 42 hours to about 60 hours, about 42 hours to about 54 hours, about 42 hours to about 48 hours, about 48 hours to about 7 days, about 48 hours to about 6 days, about 48 hours to about 5 days, about 48 hours to about 4 days, about 48 hours to about 72 hours, about 48 hours to about 66 hours, about 48 hours to about 60 hours, about 48 hours to about 54 hours, about 54 hours to about 7 days, about 54 hours to about 6 days, about 54 hours to about 5 days, about 54 hours to about 4 days, about 54 hours to about 72 hours, about 54 hours to about 66 hours, about 54 hours to about 60 hours, about 60 hours to about 7 days, about 60 hours to about 6 days, about 60 hours to about 5 days, about 60 hours to about 4 days, about 60 hours to about 72 hours, about 60 hours to about 66 hours, about 66 hours to about 7 days, about 66 hours to about 6 days, about 66 hours to about 5 days, about 66 hours to about 4 days, about 66 hours to about 72 hours, about 72 hours to about 7 days, about 72 hours to about 6 days, about 72 hours to about 5 days, about 72 hours to about 4 days, about 4 days to about 7 days, about 4 days to about 6 days, about 4 days to about 5 days, about 5 days to about 7 days, about 5 days to about 6 days, about 6 days to about 7 days; about 1 hour, about 2 hours, about 4 hours, about 6 hours, about 8 hours, about 10 hours, about 12 hours, about 18 hours, about 24 hours, about 30 hours, about 36 hours, about 42 hours, about 48 hours, about 54 hours, about 60 hours, about 66 hours, about 72 hours, about 4 days, about 5 days, about 6 days, or about 7 days) after the first time point.

### Determining Cell-Free Tumor Load in a Subject

Also provided herein are methods for determining cell-free tumor load (cfTL). In some embodiments, cfTL is detected in a biological sample isolated from the subject at a first time point. In some embodiments, cfTL is detected in a biological sample isolated from the subject at a second time point. In some embodiments, the subject has received at least one dose of the targeted therapy between the first time point and the second time point.

In some embodiments, determining cell-free tumor load (cfTL) in a subject includes detecting a first level of at least one genetic alteration present in ctDNA and/or a first level of aneuploidy in a biological sample isolated from the subject at a first time point. In some embodiments, determining cell-free tumor load (cfTL) in a subject includes detecting a first level of at least one genetic alteration present in ctDNA and/or a first level of aneuploidy in a biological sample isolated from the subject at a second time point. In some embodiments, the subject has received at least one dose of the targeted therapy between the first time point and the second time point.

In some embodiments, determining cell-free tumor load (cfTL) in a subject includes detecting the level of at least one genetic alteration (e.g., at least 2, 3, 4, 5, 6, 7, 8, 9, 10, or more genetic alterations) present in ctDNA present in a biological sample isolated from the subject. In some embodiments, no genetic alterations are detected in the subject, and determining cell-free tumor load (cfTL) in a subject includes detecting the level of aneuploidy in the subject.

In some embodiments, the subject exhibits a cfTL that is reduced at a second time point as compared to a first time point. In some embodiments, the subject exhibits a cfTL that is not reduced at a second time point as compared to a first time point.

In some embodiments, the second cfTL is not substantially reduced as compared to the first cfTL (e.g., an increase in second cfTL of less than about 10%, less than about 9%, less than about 8%, less than about 7%, less than about 6%, less than about 5%, less than about 4%, less than about 3%, less than about 2%, less than about 1%, less than about 0.5%, less than about 0.25%, less than about 0.2%, less than about 0.1%, less than about 0.05%, or less than about 0.01% as compared to the first cfTL, or a decrease of at least about 0.5%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, at least about 26%, at least about 28%, at least about 30%, at least about 40%, at least about 45%, at least about 50%, at least about 60%, at least about 65%, at least about 70%, at least about 75%, at least about 80%, at least about 85%, at least about 90%, at least about 95%, or at least about 99% in the second cfTL as compared to the first cfTL.

In some aspects, methods provided herein include obtaining from the subject additional sample(s) at additional time point(s) (e.g., at a third time point, a fourth time point, etc.) and determining whether a subject has developed resistance to a targeted therapy at the additional time point(s).

In some aspects, the second time point is about one to about four weeks (e.g., about one to about three weeks, about one to about two weeks, about two to about four weeks, about two to about three weeks, about three weeks to about four weeks; about 2 days to about 30 days, about 2 days to about 28 days, about 2 days to about 26 days, about 2 days to about 24 days, about 2 days to about 22 days, about 2 days to about 20 days, about 2 days to about 18 days, about 2 days to about 16 days, about 2 days to about 14 days, about 2 days to about 12 days, about 2 days to about 10 days, about 2 days to about 8 days, about 2 days to about 6 days, about 2 days to about 4 days, about 4 days to about 30, about 4 days to about 28 days, about 4 days to about 26 days, about 4 days to about 24 days, about 4 days to about 22 days, about 4 days to about 20 days, about 4 days to about 18 days, about 4 days to about 16 days, about 4 days to about 14 days, about 4 days to about 12 days, about 4 days to about 10 days, about 4 days to about 8 days, about 4 days to about 6 days, about 6 days to about 30, about 6 days to about 28 days, about 6 days to about 26 days, about 6 days to about 24 days, about 6 days to about 22 days, about 6 days to about 20 days, about 6 days to about 18 days, about 6 days to about 16 days, about 6 days to about 14 days, about 6 days to about 12 days, about 6 days to about 10 days, about 6 days to about 8 days, about 8 days to about 30, about 8 days to about 28 days, about 8 days to about 26 days, about 8 days to about 24 days, about 8 days to about 22 days, about 8 days to about 20 days, about 8 days to about 18 days, about 8 days to about 16 days, about 8 days to about 14 days, about 8 days to about 12 days, about 8 days to about 10 days, about 10 days to about 30, about 10 days to about 28 days, about 10 days to about 26 days, about 10 days to about 24 days, about 10 days to about 22 days, about 10 days to about 20 days, about 10 days to about 18 days, about 10 days to about 16 days, about 10 days to about 14 days, about 10 days to about 12 days, about 12 days to about 30, about 12 days to about 28 days, about 12 days to about 26 days, about 12 days to about 24 days, about 12 days to about 22 days, about 12 days to about 20 days, about 12 days to about 18 days, about 12 days to about 16 days, about 12 days to about 14 days, about 14 days to about 30, about 14 days to about 28 days, about 14 days to about 26 days, about 14 days to about 24 days, about 14 days to about 22 days, about 14 days to about 20 days, about 14 days to about 18 days, about 14 days to about 16 days, about 16 days to about 30, about 16 days to about 28 days, about 16 days to about 26 days, about 16 days to about 24 days, about 16 days to about 22 days, about 16 days to about 20 days, about 16 days to about 18 days, about 18 days to about 30, about 18 days to about 28 days, about 18 days to about 26 days, about 18 days to about 24 days, about 18 days to about 22 days, about 18 days to about 20 days, about 20 days to about 30, about 20 days to about 28 days, about 20 days to about 26 days, about 20 days to about 24 days, about 20 days to about 22 days, about 22 days to about 30, about 22 days to about 28 days, about 22 days to about 26 days, about 22 days to about 24 days, about 24 days to about 30, about 24 days to about 28 days, about 24 days to about 26 days, about 26 days to about 30, about 26 days to about 28 days, about 26 days to about 30; about 1 day, about 2 days, about 4 days, about 6 days, about 8 days, about 10 days, about 12 days, about 14 days, about 16 days, about 18 days, about 20 days, about 22 days, about 24 days, about 26 days, about 28 days, or about 30 days) after the first time point.

In some aspects, the second time point is about 1 hour to about 7 days (e.g., about 1 hour to about 6 days, about 1 hour to about 5 days, about 1 hour to about 4 days, about 1 hour to about 72 hours, about 1 hour to about 66 hours, about 1 hour to about 60 hours, about 1 hour to about 54 hours, about 1 hour to about 48 hours, about 1 hour to about 42 hours, about 1 hour to about 36 hours, about 1 hour to about 30 hours, about 1 hour to about 24 hours, about 1 hour to about 18 hours, about 1 hour to about 12 hours, about 1 hour to about 6 hours, about 1 hour to about 4 hours, about 1 hour to about 2 hours, about 2 hours to about 7 days, about 2 hours to about 6 days, about 2 hours to about 5 days, about 2 hours to about 4 days, about 2 hours to about 72 hours, about 2 hours to about 66 hours, about 2 hours to about 60 hours, about 2 hours to about 54 hours, about 2 hours to about 48 hours, about 2 hours to about 42 hours, about 2 hours to about 36 hours, about 2 hours to about 30 hours, about 2 hours to about 24 hours, about 2 hours to about 18 hours, about 2 hours to about 12 hours, about 2 hours to about 6 hours, about 2 hours to about 4 hours, about 4 hours to about 7 days, about 4 hours to about 6 days, about 4 hours to about 5 days, about 4 hours to about 4 days, about 4 hours to about 72 hours, about 4 hours to about 66 hours, about 4 hours to about 60 hours, about 4 hours to about 54 hours, about 4 hours to about 48 hours, about 4 hours to about 42 hours, about 4 hours to about 36 hours, about 4 hours to about 30 hours, about 4 hours to about 24 hours, about 4 hours to about 18 hours, about 4 hours to about 12 hours, about 4 hours to about 6 hours, about 6 hours to about 7 days, about 6 hours to about 6 days, about 6 hours to about 5 days, about 6 hours to about 4 days, about 6 hours to about 72 hours, about 6 hours to about 66 hours, about 6 hours to about 60 hours, about 6 hours to about 54 hours, about 6 hours to about 48 hours, about 6 hours to about 42 hours, about 6 hours to about 36 hours, about 6 hours to about 30 hours, about 6 hours to about 24 hours, about 6 hours to about 18 hours, about 6 hours to about 12 hours, about 12 hours to about 7 days, about 12 hours to about 6 days, about 12 hours to about 5 days, about 12 hours to about 4 days, about 12 hours to about 72 hours, about 12 hours to about 66 hours, about 12 hours to about 60 hours, about 12 hours to about 54 hours, about 12 hours to about 48 hours, about 12 hours to about 42 hours, about 12 hours to about 36 hours, about 12 hours to about 30 hours, about 12 hours to about 24 hours, about 12 hours to about 18 hours, about 18 hours to about 7 days, about 18 hours to about 6 days, about 18 hours to about 5 days, about 18 hours to about 4 days, about 18 hours to about 72 hours, about 18 hours to about 66 hours, about 18 hours to about 60 hours, about 18 hours to about 54 hours, about 18 hours to about 48 hours, about 18 hours to about 42 hours, about 18 hours to about 36 hours, about 18 hours to about 30 hours, about 18 hours to about 24 hours, about 24 hours to about 7 days, about 24 hours to about 6 days, about 24 hours to about 5 days, about 24 hours to about 4 days, about 24 hours to about 72 hours, about 24 hours to about 66 hours, about 24 hours to about 60 hours, about 24 hours to about 54 hours, about 24 hours to about 48 hours, about 24 hours to about 42 hours, about 24 hours to about 36 hours, about 24 hours to about 30 hours, about 30 hours to about 7 days, about 30 hours to about 6 days, about 30 hours to about 5 days, about 30 hours to about 4 days, about 30 hours to about 72 hours, about 30 hours to about 66 hours, about 30 hours to about 60 hours, about 30 hours to about 54 hours, about 30 hours to about 48 hours, about 30 hours to about 42 hours, about 30 hours to about 36 hours, about 36 hours to about 7 days, about 36 hours to about 6 days, about 36 hours to about 5 days, about 36 hours to about 4 days, about 36 hours to about 72 hours, about 36 hours to about 66 hours, about 36 hours to about 60 hours, about 36 hours to about 54 hours, about 36 hours to about 48 hours, about 36 hours to about 42 hours, about 42 hours to about 7 days, about 42 hours to about 6 days, about 42 hours to about 5 days, about 42 hours to about 4 days, about 42 hours to about 72 hours, about 42 hours to about 66 hours, about 42 hours to about 60 hours, about 42 hours to about 54 hours, about 42 hours to about 48 hours, about 48 hours to about 7 days, about 48 hours to about 6 days, about 48 hours to about 5 days, about 48 hours to about 4 days, about 48 hours to about 72 hours, about 48 hours to about 66 hours, about 48 hours to about 60 hours, about 48 hours to about 54 hours, about 54 hours to about 7 days, about 54 hours to about 6 days, about 54 hours to about 5 days, about 54 hours to about 4 days, about 54 hours to about 72 hours, about 54 hours to about 66 hours, about 54 hours to about 60 hours, about 60 hours to about 7 days, about 60 hours to about 6 days, about 60 hours to about 5 days, about 60 hours to about 4 days, about 60 hours to about 72 hours, about 60 hours to about 66 hours, about 66 hours to about 7 days, about 66 hours to about 6 days, about 66 hours to about 5 days, about 66 hours to about 4 days, about 66 hours to about 72 hours, about 72 hours to about 7 days, about 72 hours to about 6 days, about 72 hours to about 5 days, about 72 hours to about 4 days, about 4 days to about 7 days, about 4 days to about 6 days, about 4 days to about 5 days, about 5 days to about 7 days, about 5 days to about 6 days, about 6 days to about 7 days; about 1 hour, about 2 hours, about 4 hours, about 6 hours, about 8 hours, about 10 hours, about 12 hours, about 18 hours, about 24 hours, about 30 hours, about 36 hours, about 42 hours, about 48 hours, about 54 hours, about 60 hours, about 66 hours, about 72 hours, about 4 days, about 5 days, about 6 days, or about 7 days) after the first time point.

### Identifying the Presence or Levels of Genetic Alterations in Circulating Tumor DNA in a Subject

Provided herein are methods for identifying the presence or level of at least one genetic alteration (e.g., at least two, at least, three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least thirty-five, at least forty, at least forty-five, at least fifty, between 1 and 50, between 1 and 45, between 1 and 40, between 1 and 35, between 1 and 30, between 1 and 25, between 1 and 20, between 1 and 15, between 1 and 10, between 1 and 5, between 5 and 10, between 5 and 15, between 5 and 20, between 5 and 25, between 5 and 30, between 5 and 35, between 5 and 40, between 5 and 45, between 5 and 50, between 10 and 15, between 10 and 20, between 10 and 25, between 10 and 30, between 10 and 35, between 10 and 40, between 10 and 45, between 10 and 50, between 15 and 20, between 15 and 25, between 15 and 30, between 15 and 35, between 15 and 40, between 15 and 45, between 15 and 50, between 20 and 25, between 20 and 30, between 20 and 35, between 20 and 40, between 20 and 50, between 25 and 30, between 25 and 50, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 genetic alterations) of ctDNA in a subject (e.g., a first level of at least one genetic alteration of ctDNA at a first time point and/or a second level of at least one genetic alteration of ctDNA at a second time point).

In some embodiments, the level of at least one genetic alteration of ctDNA indicates the tumor burden in the subject.

In some embodiments, identifying the level of at least one genetic alteration of ctDNA includes identifying the presence or level of a mutation, a duplication, and/or substitution in a ctDNA sequence. In some embodiments, identifying the level of at least one genetic alteration of ctDNA includes identifying the presence or level of aneuploidy of ctDNA.

In some embodiments, the biological sample is isolated from subject. Any suitable biological sample that contains cell-free DNA can be used in accordance with any of the variety of methods disclosed herein. For example, the biological sample can include blood, plasma, serum, urine, cerebrospinal fluid, saliva, sputum, broncho-alveolar lavage, bile, lymphatic fluid, cyst fluid, stool, uterine lavage, vaginal fluids, ascites, and combinations thereof. Methods of isolating biological samples from a subject are known to those of ordinary skill in the art.

In some embodiments, the step of detecting a genetic alteration (e.g., one or more genetic alterations) in cell-free DNA is performed using one or more of the methods described herein (e.g., a targeted capture method (e.g., TEC-Seq), a next-generation sequencing method, and an array-based method, or any combinations thereof).

In some embodiments, methods provided herein can be used to detect a genetic alteration (e.g., one or more genetic alterations) in circulating tumor DNA present in cell-free DNA, where the cell-free DNA is present in an amount less than about 1500 ng, e.g., less than about 1400 ng, less than about 1300 ng, less than about 1200 ng, less than about 1100 ng, less than about 1000 ng, less than about 900 ng, less than about 800 ng, less than about 700 ng, less than about 600 ng, less than about 500 ng, less than about 400 ng, less than about 300 ng, less than about 200 ng, less than about 150 ng, less than about 100 ng, less than about 95 ng, less than about 90 ng, less than about 85 ng, less than about 80 ng, less than about 75 ng, less than about 70 ng, less than about 65 ng, less than about 60 ng, less than about 55 ng, less than about 50 ng, less than about 45 ng, less than about 40 ng, less than about 35 ng, less than about 30 ng, less than about 25 ng, less than about 20 ng, less than about 15 ng, less than about 10 ng, or less than about 5 ng. In some embodiments, methods provided herein can be used to detect a genetic alteration (e.g., one or more genetic alterations) in circulating tumor DNA present in cell-free DNA, where the circulating tumor DNA represents 100% of the cell-free DNA. In some embodiments, methods provided herein can be used to detect a genetic alteration (e.g., one or more genetic alterations) in circulating tumor DNA present in cell-free DNA, where the circulating tumor DNA represents less than 100% of the cell-free DNA, e.g. about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, about 4%, about 3%, about 2%, about 1%, about 0.95%, about 0.90%, about 0.85%, about 0.80%, about 0.75%, about 0.70%, about 0.65%, about 0.60%, about 0.55%, about 0.50%, about 0.45%, about 0.40%, about 0.35%, about 0.30%, about 0.25%, about 0.20%, about 0.15%, about 0.10%, about 0.09%, about 0.08%, about 0.07%, about 0.06%, about 0.05% of the cell-free DNA, or less.

In some embodiments, a genetic alteration and/or aneuploidy that is detected by any of the variety of methods disclosed herein is present in a cancer cell present in the subject. For example, a genetic alteration listed in Table 1 that is detected using any of the variety of methods disclosed herein can be present in a cancer cell present in the subject. In some embodiments, a genetic alteration detected by any of the variety of methods disclosed herein is confirmed to be present in a cancer cell present in the subject through further diagnostic testing (e.g., diagnostic scans, biopsies, molecular-based techniques to confirm the presence of the cancer cell mutation, or any of the other diagnostic testing methods disclosed herein or known in the art).

In some embodiments, detecting the presence or level of at least one genetic alteration of ctDNA is performed using one or more of the methods described herein (e.g., a targeted capture method, a next-generation sequencing method, and an array-based method, or any combinations thereof). In some embodiments, detecting the presence or level of ctDNA is performed using TEC-Seq, or a variation of TEC-Seq (Phallen et al., Science Transl Med, (403), 2017). For example, detecting the presence or level of at least one genetic alteration of ctDNA can include the following steps: extracting cell-free DNA from blood, ligating a low complexity pool of dual index barcode adapters to the cell-free DNA to generate a plurality of barcode adapter-ligated cell-free DNA segments, capturing the plurality of barcode adapter-ligated cell-free DNA segments, sequencing the plurality of captured barcode adapter-ligated cell-free DNA segments, aligning the sequenced plurality of captured barcode adapter-ligated cell-free DNA segments to a reference genome, and identifying sequence alterations using aligned sequences of multiple distinct molecules containing identical redundant changes. In some embodiments, the presence or level of at least one genetic alteration of ctDNA is detected (e.g., using a TEC-Seq approach) at two or more time points (e.g., a first time point prior to administration of an immunotherapy and a second time point after administration of the immunotherapy). In some embodiments, an increase in the number or level of sequence alterations (e.g., at a second time point) indicates an increase in the level of ctDNA. In some embodiments, an increase in the level of ctDNA indicates an increased tumor load or tumor burden in the subject. In some embodiments, a decrease in the number or level of sequence alterations (e.g., at a second time point) indicates a decrease in the level of ctDNA. In some embodiments, a decrease in the level of ctDNA indicates a decreased tumor load or tumor burden in the subject.

In some embodiments, detecting the presence or level of at least one genetic alteration of ctDNA is performed using sequencing technology (e.g., a next-generation). A variety of sequencing technologies are known in the art. For example, a variety of technologies for detection and characterization of circulating tumor DNA in cell-free DNA is described in Haber and Velculescu, Blood-Based Analyses of Cancer: Circulating Tumor Cells and Circulating Tumor DNA, Cancer Discov., June; 4(6):650-61. doi: 10.1158/2159-8290.CD-13-1014, 2014, incorporated herein by reference in its entirety. Non-limiting examples of such techniques include SafeSeqs (Kinde et. al, Detection and quantification of rare mutations with massively parallel sequencing, Proc Natl Acad Sci USA; 108, 9530-5, 2011), OnTarget (Forshew et al., Noninvasive identification and monitoring of cancer mutations by targeted deep sequencing of plasma DNA, Sci Transl Med; 4:136ra68, 2012), and TamSeq (Thompson et al., Winnowing DNA for rare sequences: highly specific sequence and methylation based enrichment. PLoS ONE, 7:e31597, 2012), each of which is incorporated herein by reference in its entirety.

In some embodiments, detecting the presence or level of at least one genetic alteration of ctDNA is performed using droplet digital PCR (ddPCR). In some embodiments, detecting the presence or level of at least one genetic alteration of ctDNA is performed using other sequencing technologies, including but not limited to, chain-termination techniques, shotgun techniques, sequencing-by-synthesis methods, methods that utilize microfluidics, other capture technologies, or any of the other sequencing techniques known in the art that are useful for detection of small amounts of DNA in a sample (e.g., circulating tumor DNA in a cell-free DNA sample).

In some embodiments, detecting the presence or level of at least one genetic alteration of ctDNA is performed using array-based methods. For example, detecting the presence or level of at least one genetic alteration of ctDNA can be performed using a DNA microarray. In some embodiments, a DNA microarray can detect the presence or level of at least one genetic alteration of ctDNA. In some embodiments, cell-free DNA is amplified prior to detecting the presence or level of at least one genetic alteration of ctDNA. Non-limiting examples of array-based methods that can be used in any of the methods described herein, include: a complementary DNA (cDNA) microarray (Kumar et al. (2012) J. Pharm. Bioallied Sci. 4(1): 21-26; Laere et al. (2009) Methods Mol. Biol. 512: 71-98; Mackay et al. (2003) Oncogene 22: 2680-2688; Alizadeh et al. (1996) Nat. Genet. 14: 457-460), an oligonucleotide microarray (Kim et al. (2006) Carcinogenesis 27(3): 392-404; Lodes et al. (2009) PLoS One 4(7): e6229), a bacterial artificial chromosome (BAC) clone chip (Chung et al. (2004) Genome Res. 14(1): 188-196; Thomas et al. (2005) Genome Res. 15(12): 1831-1837), a single-nucleotide polymorphism (SNP) microarray (Mao et al. (2007) Curr. Genomics 8(4): 219-228; Jasmine et al. (2012) PLoS One 7(2): e31968), a microarray-based comparative genomic hybridization array (array-CGH) (Beers and Nederlof (2006) Breast Cancer Res. 8(3): 210; Pinkel et al. (2005) Nat. Genetics 37: S11-S17; Michels et al. (2007) Genet. Med. 9: 574-584), a molecular inversion probe (MIP) assay (Wang et al. (2012) Cancer Genet 205(7-8): 341-55; Lin et al. (2010) BMC Genomics 11: 712). In some embodiments, the cDNA microarray is an Affymetrix microarray (Irizarry (2003) Nucleic Acids Res 31:e15; Dalma-Weiszhausz et al. (2006) Methods Enzymol. 410: 3-28), a NimbleGen microarray (Wei et al. (2008) Nucleic Acids Res 36(9): 2926-2938; Albert et al. (2007) Nat. Methods 4: 903-905), an Agilent microarray (Hughes et al. (2001) Nat. Biotechnol. 19(4): 342-347), or a BeadArray array (Liu et al. (2017) Biosens Bioelectron 92: 596-601). In some embodiments, the oligonucleotide microarray is a DNA tiling array (Mockler and Ecker (2005) Genomics 85(1): 1-15; Bertone et al. (2006) Genome Res 16(2): 271-281). Other suitable array-based methods are known in the art.

### Selecting a Subject for Further Diagnostic Testing

Also provided herein are methods for selecting a subject for further diagnostic testing. In some embodiments, methods for selecting a subject for further diagnostic testing include detecting cfTL (e.g., one or more genetic alterations and/or aneuploidy) in cell-free DNA in a biological sample isolated from the subject, and selecting a subject for further diagnostic testing when identified sufficiently high cfTL is detected.

In some embodiments, the biological sample is isolated from subject. Any suitable biological sample that contains cell-free DNA can be used in accordance with any of the variety of methods disclosed herein. For example, the biological sample can include blood, plasma, serum, urine, cerebrospinal fluid, saliva, sputum, broncho-alveolar lavage, bile, lymphatic fluid, cyst fluid, stool, uterine lavage, vaginal fluids, ascites, and combinations thereof. Methods of isolating biological samples from a subject are known to those of ordinary skill in the art.

In some embodiments, the step of detecting a genetic alteration (e.g., one or more genetic alterations) in cell-free DNA is performed using one or more of the methods described herein (e.g., a targeted capture method, a next-generation sequencing method, and an array-based method, or any combinations thereof).

In some embodiments, methods provided herein for selecting a subject for further diagnostic testing include detecting cfTL (e.g., one or more genetic alterations and/or aneuploidy) in circulating tumor DNA present in cell-free DNA, where the cell-free DNA is present in an amount less than about 1500 ng, e.g., less than about 1400 ng, less than about 1300 ng, less than about 1200 ng, less than about 1100 ng, less than about 1000 ng, less than about 900 ng, less than about 800 ng, less than about 700 ng, less than about 600 ng, less than about 500 ng, less than about 400 ng, less than about 300 ng, less than about 200 ng, less than about 150 ng, less than about 100 ng, less than about 95 ng, less than about 90 ng, less than about 85 ng, less than about 80 ng, less than about 75 ng, less than about 70 ng, less than about 65 ng, less than about 60 ng, less than about 55 ng, less than about 50 ng, less than about 45 ng, less than about 40 ng, less than about 35 ng, less than about 30 ng, less than about 25 ng, less than about 20 ng, less than about 15 ng, less than about 10 ng, or less than about 5 ng. In some embodiments, methods provided herein for selecting a subject for further diagnostic testing include detecting cfTL (e.g., one or more genetic alterations and/or aneuploidy) in circulating tumor DNA present in cell-free DNA, where the circulating tumor DNA represents 100% of the cell-free DNA. In some embodiments, methods provided herein for selecting a subject for further diagnostic testing include detecting cfTL (e.g., one or more genetic alterations and/or aneuploidy) in circulating tumor DNA present in cell-free DNA, where the circulating tumor DNA represents less than 100% of the cell-free DNA, e.g. about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, about 4%, about 3%, about 2%, about 1%, about 0.95%, about 0.90%, about 0.85%, about 0.80%, about 0.75%, about 0.70%, about 0.65%, about 0.60%, about 0.55%, about 0.50%, about 0.45%, about 0.40%, about 0.35%, about 0.30%, about 0.25%, about 0.20%, about 0.15%, about 0.10%, about 0.09%, about 0.08%, about 0.07%, about 0.06%, about 0.05% of the cell-free DNA, or less.

In some embodiments, the diagnostic testing method is a scan. In some embodiments, the scan is a computed tomography (CT), a CT angiography (CTA), a esophagram (a Barium swallom), a Barium enema, a magnetic resonance imaging (MRI), a PET scan, an ultrasound (e.g., an endobronchial ultrasound, an endoscopic ultrasound), an X-ray, a DEXA scan.

In some embodiments, the diagnostic testing method is a physical examination, such as an anoscopy, a bronchoscopy (e.g., an autofluorescence bronchoscopy, a white-light bronchoscopy, a navigational bronchoscopy), a colonoscopy, a digital breast tomosynthesis, an endoscopic retrograde cholangiopancreatography (ERCP), an ensophagogastroduodenoscopy, a mammography, a Pap smear, a pelvic exam, a positron emission tomography and computed tomography (PET-CT) scan.

In some embodiments, the diagnostic testing method is a biopsy (e.g., a bone marrow aspiration, a tissue biopsy). In some embodiments, the biopsy is performed by fine needle aspiration or by surgical excision. In some embodiments, the diagnostic testing methods further includes obtaining a biological sample (e.g., a tissue sample, a urine sample, a blood sample, a check swab, a saliva sample, a mucosal sample (e.g., sputum, bronchial secretion), a nipple aspirate, a secretion or an excretion).

In some embodiments, the diagnostic testing method includes determining the presence of a circulating tumor cell. In some embodiments, the diagnostic testing method includes determining the complete blood cell count (i.e. the percentage and types of immune cells). In some embodiments, the diagnostic testing method is a fecal occult blood test.

For example, a subject selected for further diagnostic testing can also be selected for increased monitoring, in which the subject is administered a diagnostic test at a frequency of twice daily, daily, bi-weekly, weekly, bi-monthly, monthly, quarterly, semi-annually, annually, or any at frequency therein. In some embodiments, a subject selected for further diagnostic testing can also be selected for increased monitoring, in which the subject is administered one or more additional diagnostic tests compared to a subject that has not been selected for further diagnostic testing and increased monitoring.

### Targeted Therapy

As used herein, the terms “targeted therapy” or “molecularly targeted therapy” refer to a treatment that recognize and bind to cell-surface proteins, secreted proteins, peptides, or combinations thereof, that are associated with cancer cells and/or cancer-associated cancer cells, without harming non-cancer cells (e.g., healthy cells). In some embodiments of any of the methods described herein, the subject has received a targeted therapy.

In some embodiments, a targeted therapy has a cytotoxic effect on a cancer cell. In other embodiments, a targeted therapy has a cytostatic effect on a cancer cell. In some embodiments, a targeted therapy is an antibody (e.g., a monoclonal antibody, a humanized chimeric antibody), an antigen-binding fragment thereof, a small molecule, a small molecule drug conjugate, a small inhibitory nucleic acid, or a combination thereof.

In some embodiments, a targeted therapy is a hormone therapy, a signal transduction inhibitor, a gene expression modulator, an inducer of apoptosis, an inhibitor of angiogenesis, or a kinase inhibitor. In some embodiments, the kinase inhibitor is a tyrosine kinase inhibitor (TKI). In some embodiments, the kinase inhibitor is a janus kinase inhibitor, an ALK inhibitor, a Bcl-2 inhibitor, a PARP inhibitor, a PI3K inhibitor, a Braf inhibitor, a MEK inhibitor, or a CDK inhibitor.

In some embodiments, the targeted therapy is an anti-angiogenic agent (e.g., bevacizumab (avastin), ramucirumab (cyramza)).

In some embodiments, the targeted therapy is an EGFR inhibitor (e.g., erlotinib (tarceva), afatinib (gilotrif), gefitinib (iressa), necitumumab (portrazza), cetuximab, osimertinib (AZD9291, Tagrisso), rociletinib (CO-1686), HM61713 (BI 1482694), ASP8273, EGF816, PF-06747775). See, e.g., Wang et al. (2016) J. Hematol Oncol 9:34; Cross et al. (2014) Cancer Discov. 4(9): 1046-61; Walter et al. (2013) Cancer Discov 3(12): 1404-15; Park et al. (2015) ASCO Meeting Abstract 33(15): 8084; Sequist et al. (2015) 372(18): 1700-9; Lee et al. (2014) Cancer Res 74(19Supplement): LB-100; Sakagami et al. (2014) Cancer Res 74(19 Supplement): 1728; Goto et al. (2015) ASCO Meeting Abstract 33(15_Suppl):8014; Jia et al. (2016) Cancer Res 76: 1591-602.

In some embodiments, the targeted therapy is an ALK inhibitor (e.g., crizotinib (xalkori), ceritinib (zykadia, LDK378), alectinib (alecensa, RO5424802; CH5424802), brigatinib (alunbrig, AP26113), lorlatinib (PF-06463922), TSR-011, RXDX-101 (NMS-E628), X-396, CEP-37440). See, e.g., Tartarone et al. (2017) Med. Oncol. 34(6): 110; Galkin et al. (2007) PNAS 104(1): 270-275; Friboulet et al. (2014) Cancer Discov. 4(6); 662-73; Chen et al. (2013) 56(14): 5673-5674; Shaw et al. (2014) N. Engl. J. Med. 370(13): 1189-1197; Sakamoto et al. (2011) Cancer Cell 19(5): 679-690; Squillace et al. (2013) Cancer Res. 73(8_suppl_: 5655; Mori et al. (2014) 13(2): 329-340; Patnaik et al. (2013) J. Clin. Oncol. 31 (15 suppl); Weiss et al. (2013) J Thorac Oncol. 8(suppl2): S618; Ardini et al. (2009) Mol. Cancer Ther. 8(12suppl): A244; Horn et al. (2014) J. Clin. Oncol. 32(15suppl); Cheng et al. (2012) Mol. Cancer Ther. 11(3): 670-679; Zhang et al. (2011) Cancer Res. 70(8suppl): LB-298; Awad and Shaw (2014) Clin. Adv. Hematol. Oncol. 12(7): 429-439.

In some embodiments, the targeted therapy is a heat shock protein 90 inhibitor (e.g, AUY922, ganetspib, AT13387). See, e.g., Pillai et al. (2014) Curr Opin Oncol. 26(2): 159-164; Normant et al. (2011) Oncogene 30(22): 2581-2586; Sequist et al. (2010) J. Clin. Oncol. 28(33): 4953-4960; Sang et al. (2013) Cancer Discov. 3(4): 430-443; Felip et al. (2012) Ann Oncol 23(suppl9); Miyajima et al. (2013) Cancer Res. 73(23): 7022-7033.

In some embodiments, the targeted therapy is a RET inhibitor (e.g., cabozantinib (XL184), vandetanib, alectinib, sorafenib, sunitinib, ponatinib) See, e.g., Drilon et al. (2013) Cancer Discov 3:6305; Gautschi et al. (2013) J. Thorac Oncol 8: e43-4; Kodama et al. (2014) Mol. Cancer Ther. 13: 2910-8; Lin et al. (2016) J. Thoracic Oncol. 11(11): 2027-2032; Rosell and Karachaliou (2016) Lancet 17(12): 1623-1625; Falchook et al. (2016) J. Clin Oncol. 34(15): e141-144; Shaw et al. (2013) Nat Rev Cancer 13: 772-787; Gozgit et al. (2013) Cancer Res 73 (Suppl. 1): 2084.

In some embodiments, the targeted therapy is a BRAF inhibitor (e.g., dabrafenib, vemurafenib). See, e.g., Planchard et al. (2013) J. Clin. Oncol. 31:8009; Gautschi et al. (2013) Lung Cancer 82: 365-367; Schmid et al. (2015) Lung Cancer 87: 85-87.

In some embodiments, the targeted drug therapy is a MET inhibitor (e.g., onartuzumab, ficlatuzumab, rilotumumab, tivantinib, crizotinib). See, e.g., Spigel et al. (2014) J. Clin. Oncol. 32: 8000; Patnail et al. (2014) Br. J. Cancer 111: 272-280; Gordon et al. (2010): Clin. Cancer Res. 16: 699-710; Sequist et al. (2011) J. Clin. Oncol. 29: 3307-3315; Zou et al. (2007) Cancer Res. 67: 4408-4417; Ou et al. (2011) J. Thorac. Oncol. 6: 942-946.

Non-limiting examples of tyrosine kinase inhibitors include: imatinib, sorafenib, sunitinib, dasatinib, lapatinib, nilotinib, bortezomib, axitinib, pazopanib, afatinib, crizotinib, erlotinib, gefitinib, and osimertinib.

Various other examples of targeted therapies are known in the art.

### Therapeutic Interventions

In some embodiments, when a targeted therapy is determined not to be effective in a subject (e.g., using any of the variety of methods disclosed herein), a therapeutic intervention (e.g., a therapeutic intervention that is different from the ineffective targeted therapy) can be administered to the subject. Exemplary therapeutic interventions include, without limitation, adoptive T cell therapy (e.g., chimeric antigen receptors and/or T cells having wild-type or modified T cell receptors), chimeric antigen receptor (CAR) T cell therapy, radiation therapy, surgery (e.g., surgical resection), and administration of one or more chemotherapeutic agents, administration of immune checkpoint inhibitors, targeted therapies such as kinase inhibitors (e.g., kinase inhibitors that target a particular genetic lesion, such as a translocation or mutation), signal transduction inhibitors, bispecific antibodies, and/or monoclonal antibodies. Such therapeutic interventions can be administered alone or in combination.

In some embodiments, the therapeutic intervention can include an immune checkpoint inhibitor (e.g., a single immune checkpoint inhibitor or a combination of immune checkpoint inhibitors). Non-limiting examples of immune checkpoint inhibitors include nivolumab (Opdivo), pembrolizumab (Keytruda), atezolizumab (tecentriq), avelumab (bavencio), durvalumab (imfinzi), ipilimumab (yervoy). See, e.g., Pardoll (2012) Nat. Rev Cancer 12: 252-264; Sun et al. (2017) Eur Rev Med Pharmacol Sci 21(6): 1198-1205; Hamanishi et al. (2015) J. Clin. Oncol. 33(34): 4015-22; Brahmer et al. (2012) N Engl J Med 366(26): 2455-65; Ricciuti et al. (2017) J. Thorac Oncol. 12(5): e51-e55; Ellis et al. (2017) Clin Lung Cancer pii: S1525-7304(17)30043-8; Zou and Awad (2017) Ann Oncol 28(4): 685-687; Sorscher (2017) N Engl J Med 376(10: 996-7; Hui et al. (2017) Ann Oncol 28(4): 874-881; Vansteenkiste et al. (2017) Expert Opin Biol Ther 17(6): 781-789; Hellmann et al. (2017) Lancet Oncol. 18(1): 31-41; Chen (2017) J. Chin Med Assoc 80(1): 7-14.

In some embodiments, a therapeutic intervention is adoptive T cell therapy (e.g., chimeric antigen receptors and/or T cells having wild-type or modified T cell receptors). See, e.g., Rosenberg and Restifo (2015) Science 348(6230): 62-68; Chang and Chen (2017) Trends Mol Med 23(5): 430-450; Yee and Lizee (2016) Cancer J. 23(2): 144-148; Chen et al. (2016) Oncoimmunology 6(2): e1273302; US 2016/0194404; US 2014/0050788; US 2014/0271635; U.S. Pat. No. 9,233,125; incorporated by reference in their entirety herein.

In some embodiments, a therapeutic intervention is a chemotherapeutic agent. Non-limiting examples of chemotherapeutic agents include: amsacrine, azacitidine, axathioprine, bevacizumab (or an antigen-binding fragment thereof), bleomycin, busulfan, carboplatin, capecitabine, chlorambucil, cisplatin, cyclophosphamide, cytarabine, dacarbazine, daunorubicin, docetaxel, doxifluridine, doxorubicin, epirubicin, erlotinib hydrochlorides, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, all-trans retinoic acid, streptozocin, tafluposide, temozolomide, teniposide, tioguanine, topotecan, uramustine, valrubicin, vinblastine, vincristine, vindesine, vinorelbine, and combinations thereof. Additional examples of anti-cancer therapies are known in the art; see, e.g. the guidelines for therapy from the American Society of Clinical Oncology (ASCO), European Society for Medical Oncology (ESMO), or National Comprehensive Cancer Network (NCCN).

In some embodiments, the therapeutic intervention can result in an early onset of remission of a cancer in a subject. In some embodiments, the therapeutic intervention can result in an increase in the time of remission of a cancer in a subject. In some embodiments, the therapeutic intervention can result in an increase in the time of survival of a subject. In some embodiments, the therapeutic intervention can result in decreasing the size of a solid primary tumor in a subject. In some embodiments, the therapeutic intervention can result in decreasing the volume of a solid primary tumor in a subject. In some embodiments, the therapeutic intervention can result in decreasing the size of a metastasis in a subject. In some embodiments, the therapeutic intervention can result in decreasing the volume of a metastasis in a subject. In some embodiments, the therapeutic intervention can result in decreasing the tumor burden in a subject.

In some embodiments, the therapeutic intervention can result in improving the prognosis of a subject. In some embodiments, the therapeutic intervention can result in decreasing the risk of developing a metastasis in a subject. In some embodiments, the therapeutic intervention can result in decreasing the risk of developing an additional metastasis in a subject. In some embodiments, the therapeutic intervention can result in decreasing cancer cell migration in a subject. In some embodiments, the therapeutic intervention can result in decreasing cancer cell invasion in a subject. In some embodiments, the therapeutic intervention can result in a decrease in the time of hospitalization of a subject. In some embodiments, the therapeutic intervention can result in a decrease of the presence of cancer stem cells within a tumor in a subject.

In some embodiments, the therapeutic intervention can result in an increase in immune cell infiltration within the tumor microenvironment in a subject. In some embodiments, the therapeutic intervention can result in altering the immune cell composition within the tumor microenvironment of a tumor in a subject. In some embodiments, the therapeutic intervention can result in modulating a previously-immunosuppressive tumor microenvironment into an immunogenic, inflammatory tumor microenvironment. In some embodiments, the therapeutic intervention can result in a reversal of the immunosuppressive tumor microenvironment in a subject.

In some embodiments, the therapeutic intervention can halt tumor progression in a subject. In some embodiments, the therapeutic intervention can delay tumor progression in a subject. In some embodiments, the therapeutic intervention can inhibit tumor progression in a subject. In some embodiments, the therapeutic intervention can inhibit immune checkpoint pathways of a tumor in a subject. In some embodiments, the therapeutic intervention can immuno-modulate the tumor microenvironment of a tumor in a subject. In some embodiments, the therapeutic intervention can immuno-modulate the tumor macroenvironment of a tumor in a subject.

In some embodiments of any of the methods described herein, the subject can be administered a single or multiple doses (e.g., two, three, four, five, six, seven, eight, nine, or ten doses) of any of the therapeutic interventions described herein.

In some embodiments of any of the methods described herein, the method can further include administering one or more therapeutic interventions.

The term “immunotherapy” refers to a therapeutic treatment that involves administering to a patient an agent that modulates the immune system. For example, an immunotherapy can increase the expression and/or activity of a regulator of the immune system. In other instances, an immunotherapy can decrease the expression and/or activity of a regulator of the immune system. In some instances, an immunotherapy can recruit and/or enhance the activity of an immune cell. An example of an immunotherapy is a therapeutic treatment that involves administering at least one, e.g., two or more, immune checkpoint inhibitors. Exemplary immune checkpoint inhibitors are CTLA-4 inhibitors, PD-1 inhibitors or PD-L1 inhibitors, or combinations thereof.

In some instances, the immunotherapy is an immune checkpoint inhibitor. For example, the immunotherapy can include administering one or more immune checkpoint inhibitors. In some embodiments, the immune checkpoint inhibitor is a CTLA-4 inhibitor, a PD-1 inhibitor or a PD-L1 inhibitor. An exemplary CTLA-4 inhibitor would be, e.g., ipilimumab (Yervoy®) or tremelimumab (CP-675,206). In some embodiments, the PD-1 inhibitor is pembrolizumab (Keytruda®) or nivolumab (Opdivo®). In some embodiments, the PD-L1 inhibitor is atezolizumab (Tecentriq®), avelumab (Bavencio®) or durvalumab (Imfinzi™) As used herein, the terms “in combination” or “combination therapy” describe any concurrent or parallel treatment with at least two distinct therapeutic agents, e.g., administration of any of at least two therapeutic interventions. In some embodiments of any of the methods described herein, the one or more therapeutic interventions are administered sequentially or simultaneously to the subject after the cancer cell has been detected. For example, the one or more therapeutic interventions can include chemotherapeutic agents, anti-angiogenic agents, apoptosis-inducing agents, surgical resection, and radiotherapy. In some embodiments, combined therapy is an epigenetic therapy (e.g., any of the epigenetic therapies described herein) and an immunotherapy (e.g., any of the immunotherapies described herein). In some embodiments, the combined therapy is 5-AZA and an immune checkpoint inhibitor (e.g., anti-PD1 and/or anti-CTLA-4 inhibitor) (Kim (2014) PNAS 111(32): 11774-1179; Wang (2015) Cancer Immunol. Res. 3(9): 1030-1041; Juergens et al. (2011) Cancer Discov 1(7): 598-607).

### Cancers

A subject according to any of the methods described herein can have a cancer that includes, without limitation, lung cancer (e.g., small cell lung carcinoma or non-small cell lung carcinoma), papillary thyroid cancer, medullary thyroid cancer, differentiated thyroid cancer, recurrent thyroid cancer, refractory differentiated thyroid cancer, lung adenocarcinoma, bronchioles lung cell carcinoma, multiple endocrine neoplasia type 2A or 2B (MEN2A or MEN2B, respectively), pheochromocytoma, parathyroid hyperplasia, breast cancer, colorectal cancer (e.g., metastatic colorectal cancer), papillary renal cell carcinoma, ganglioneuromatosis of the gastroenteric mucosa, inflammatory myofibroblastic tumor, or cervical cancer, acute lymphoblastic leukemia (ALL), acute myeloid leukemia (AML), cancer in adolescents, adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, colon cancer, colorectal cancer, craniopharyngioma, cutaneous T-cell lymphoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, myelogenous leukemia, myeloid leukemia, multiple myeloma, myeloproliferative neoplasms, nasal cavity and paranasal sinus cancer, nasopharyngeal cancer, neuroblastoma, non-Hodgkin's lymphoma, non-small cell lung cancer, oral cancer, oral cavity cancer, lip cancer, oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromosytoma, pituitary cancer, plasma cell neoplasm, pleuropulmonary blastoma, pregnancy and breast cancer, primary central nervous system lymphoma, primary peritoneal cancer, prostate cancer, rectal cancer, renal cell cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis and ureter, unknown primary carcinoma, urethral cancer, uterine cancer, uterine sarcoma, vaginal cancer, vulvar cancer, Waldenstrom Macroglobulinemia, and Wilms' tumor.

In some embodiments of any of the methods described herein, the subject has non-small cell lung cancer, melanoma, colorectal cancer, ovarian cancer, or breast cancer.

In some embodiments of any of the methods described herein, the subject has the cancer is selected from the group consisting of: a head and neck cancer, a central nervous system cancer, a lung cancer, a mesothelioma, an esophageal cancer, a gastric cancer, a gall bladder cancer, a liver cancer, a pancreatic cancer, a melanoma, an ovarian cancer, a small intestine cancer, a colorectal cancer, a breast cancer, a sarcoma, a kidney cancer, a bladder cancer, a uterine cancer, a cervical cancer, and a prostate cancer. Various embodiments of such cancers, as therapeutic interventions appropriate to treat such cancers, are described herein.

### Colorectal Cancer

In some embodiments wherein the subject has colorectal cancer, the subject may have hereditary colorectal cancer. In some embodiments, the subject has polyposis (e.g., familial adenomatous polyposis (FAP) or attenuated FAP (AFAP) (Half et al. (2009) Orphanet J Rare Dis. 4:22; Knudsen et al. (2003) Fam Cancer 2:43-55). In some embodiments, the subject has a mutation in an adenomatosis polyposis coli (APC) gene and/or a mutY DNA glycosylase (MYH) gene (Theodoratou et al. (2010) Br. J. Cancer 103: 1875-1884). In some embodiments, the subject has hereditary nonpolyposis colorectal cancer (HNPCC; also known as Lynch Syndrome) (Marra et al. (1995) J. Natl. Cancer Inst 87: 1114-1135). In some embodiments, the subject has a mutation in a DNA mismatch repair gene (e.g., mutL homolog 1 (MLHI), mutS homolog 2 (MSH2), mutS homolog 6 (MSH6) and/or PMS1 homolog 2 (PMS2)). In some embodiments, the subject has a mutation in an epithelial cell adhesion molecule (EPCAM) gene. In some embodiments, the subject has a mutation in an axin-related protein 2 (AXIN2) gene (Lammi et al. (2004) Am. J. Hum. Genet. 74: 1043-1050). In some embodiments wherein a colorectal cancer cell has been detected in the subject, the subject has oligopolyposis, juvenile polyposis syndrome, Cowden syndromw, Peutz-Jeghers syndrome (Giardiello et al. (2006) Clin. Gastroenterol. Hepatol. 4:408-415), or serrated polyposis syndrome (Torlakovic et al. (1996) Gastroenterology 110: 748-755). In some embodiments, the subject has hereditary mixed polyposis syndrome (Whitelaw et al. (1997) Gastroenterology 112: 327-334; Tomlinson et al. (1999) Gastronenterology 116: 789-795).

In some embodiments wherein the subject has colorectal cancer, the subject has a colorectal cancer that has at least one mutation in a gene selected from the group consisting of: adenomatosis polyposis coli (APC), mutY DNA glycosylase (MYH), mutL homolog 1 (MLHI), mutS homolog 2 (MSH2), mutS homolog 6 (MSH6), PMS1 homolog 2 (PMS2), epithelial cell adhesion molecule (EPCAM), DNA polymerase epsilon (POLE), DNA polymerase delta 1 (POLD1), nth like DNA glycosylase 1 (NTHL1), bone morphogenetic protein receptor type 1A (BMPRIA), SMAD family member 4 (SMAD4), phosphatase and tensin homolog (PTEN), serine/threonine kinase 11 (LKB1, STKI1), transforming growth factor beta receptor 2 (TGFβRI1), phosphatidylinositol-4,5-biphosphate-3-kinase catalytic subunit alpha (PIK3CA), tumor protein p53 (TP53), epidermal growth factor receptor (EGFR), B-raf proto-oncogene (BRAF), phosphatidylinositol-4,5-biphosphate-3-kinase (PI3K), A-T rich interaction domain 1A (ARIDIA), sex determining region Y-bod 9 (SOX9), erb-b2 receptor tyrosine kinase 2 (ERBB2), insulin like growth factor 2 (IGF2), APC membrane recruitment protein (FAM123B; AMER1), neuron navigator 2 (NAV2), vacuolar protein sorting 72 homolog (TCFL1; VPS72), N-Ras proto-oncogene (NRAS), and combinations thereof. See, e.g., Armaghany et al. (2012) Gastrointest. Cancer Res. 5(1): 19-27; Bulow et al. (2004) Gut 53: 381-386; Zeichner et al. (2012) Clin. Med. Insights Oncol. 6: 315-323; The Cancer Genomic Atlas Network (2012) Nature 487: 330-337; Kemp et al. (2004) Hum. Mol. Genet. 13(suppl_2: R177-R185; Zouhairi et al. (2011) Gastrointest Cancer Res 4(1): 15-21.

In some embodiments, the subject has a genetic mutation that can result in activation of a proto-oncogene (e.g., KRAS). In some embodiments, the subject has a genetic mutation that can result in inactivation of a tumor suppressor gene (e.g., 1, 2, 3, 4, 5, 6, at least 1, at least 2 or at least 3 tumor suppressor genes). In some embodiments, at least three tumor suppressor genes are inactivated (e.g., APC, TP53, and loss of heterozygosity of long arm of chromosome 18). In some embodiments, the subject has a genetic mutation in a gene involved in the APC/Wnt/β-catenin pathway. In some embodiments, the genetic mutation is a nonsense mutation or a frameshift mutation, thereby resulting in a truncated protein. In some embodiments, the genetic mutation causes microsatellite instability, epigenetic instability and/or aberrant CpG methylation.

In some embodiments of any of the methods described herein wherein the subject has previously been diagnosed with colorectal cancer, the subject is administered an additional therapeutic intervention that specifically targets the genetic modifications present in the subject's colorectal cancer. In some embodiments, the subject was previously administered an anti-EGFR monoclonal antibody (e.g., cetuximab or panitumumab) (Cunningham et al. (2004) N. Engl. J. Med. 351(4): 337-345). In some embodiments, the therapeutic invention is an antiangiogenic agent. In some embodiments, the antiangiogenic agent is bevacizumab (Avastin) (Hurwitz et al. (2004) N. Engl. J. Med. 350: 2335-2342). In some embodiments, the antiangiogenic agent is a VEGF inhibitor (e.g., aflibercept (Tang et al. (2008) J. Clin. Oncol 26 (May 20 suppl; abstr 4027); vatalanib (PTK/ZK222584; Hecht et al. (2005) ASCO Annual Meeting Proceedings J. Clin. Oncol. 23: 16S (abstr. LBA3)); sunitinib (Saltz et al. (2007) J. Clin. Oncol. 25: 4793-4799); AZD2171 (Rosen et al. (2007) J. Clin. Oncol. 25: 2369-76); AMG 706 (Drevis et al. (2007) 25: 3045-2054)).

Non-limiting examples of chemotherapy treatments that can be used in a subject with colorectal cancer include: 5-FU, leucovorin, oxaliplatin (Eloxatin), capecitabine, celecoxib and sulindac. In some embodiments, a combination of chemotherapeutic agents is used, e.g., FOLFOX (5-FU, leucovorin and oxaliplatin), FOLFIRI (leucovorin, 5-FU and irinotecan (Camptosar), CapeOx (capecitabine (Xeloda) and oxaliplatin). In some embodiments, the therapeutic intervention is a mammalian target of rapamycin (mTOR) inhibitor (e.g., a rapamycin analog (Kesmodel et al. (2007) Gastrointestinal Cancers Symposium (abstr 234)); RAD-001 (Tabernero et al. (2008) J. Clin. Oncol. 26: 1603-1610). In some embodiments, the therapeutic intervention is a protein kinase C antagonist (e.g., enzastaurin (Camidge et al. (2008) Anticancer Drugs 19:77-84, Resta et al. (2008) J. Clin. Oncol. 26 (May 20 suppl) (abstr 3529)). In some embodiments, the therapeutic intervention is an inhibitor of nonreceptor tyrosine kinase Src (e.g., AZ0530 (Tabernero et al. (2007) J. Clin. Oncol. 25: 18S (abstr 3520))). In some embodiments, the therapeutic intervention is an inhibitor of kinesin spindle protein (KSP) (e.g., ispinesib (SB-715992) (Chu et al. (2004) J. Clin. Oncol. 22:14S (abstr 2078), Burris et al. (2004) J. Clin. Oncol. 22: 128 (abstr 2004))).

In some embodiments, the therapeutic intervention is surgery (e.g., polypectomy, partial colectomy, colectomy or diverting colostomy). In some embodiments, adjuvant chemotherapy is further administered to the subject after surgery (e.g., polypectomy or partial colectomy). In some embodiments, the therapeutic intervention is a prophylactic surgery (e.g., colectomy). In some embodiments, a cancer may be removed by ablation or embolization.

### Ovarian Cancer

In some embodiments of any of the methods described herein, the subject may have hereditary ovarian cancer (Petrucelli et al. (2010) Gen. Med 12:245-259). In some embodiments, the subject has another genetic condition that may cause ovarian cancer (e.g., Lynch syndrome, Peutz-Jeghers syndrome, nevoid basal cell carcinoma syndrome (NBCCS; also known as Gorlin syndrome), Li-Fraumeni syndrome or Ataxia-Telangiecstasia (Cancer.Net). In some embodiments, the subject may have an invasive epithelial ovarian cancer, an epithelial tumor of low malignant potential (also known as an atypical proliferating tumor or a borderline tumor), a germ cell tumor of the ovary (e.g. a malignant germ cell tumor, a dysgerminoma, an immature teratoma) or a stromal tumor of the ovary.

In some embodiments, the subject's ovarian cancer was caused by a somatic mutation in a gene. In some embodiments, the subject has a mutation in a gene selected from the group consisting of: tumor protein p53 (TP53), breast cancer 1 (BRCA1), breast cancer 2 (BRCA2), mutL homolog 1 (MLHI), mutS homolog 2 (MSH2), AKT serine/threonine kinase 1 (AKTI), BRAC1 associated ring domain 1 (BARD1), BRAC1 interacting protein C-terminal helicase 1 (BRIP1), epithelial cadherin 1 (CDH1), checkpoint kinase 2 (CHEK2), catenin beta 1 (CTNNB1), MRE11 homolog (MRE11), mutS homolog 6 (MSH6), nibrin (NBN), opiod binding protein/cell adhesion molecule like (OPCML), partner and localizer of BRCA2 (PALB2), phosphatidylinositol-4,5-biphosphate-3-kinase catalytic subunit alpha (PIK3CA), PMS1 homolog 2 (PMS2), parkin RBR E3 ubiquitin protein ligase (PRKN), RAD50 double strand break repair protein (RAD50), RAD51 recombinase (RAD51), serine/threonine kinase 11 (LKB1, STKI1), neurofibromin (NF1), retinoblastoma 1 (RB1), cyclin dependent kinase 12 (CDK12), and combinations thereof. See, e.g., Kurman et al. (2011) Hum. Pathol. 42(7): 918-31; Nakayama et al. (2006) Cancer Biol. Ther. 5(7): 779-785; Singer et al. (2003) J. Natl Cancer Inst 95(6): 484-6; Kuo et al. (2009) Am. J. Pathol. 174(5): 1597-601; Gemignani et al. (2003) Gynecol. Oncol. 90(2): 378-81; Levine et al. (2005) Clin. Cancer Res. 11(8): 2875-8; Wang et al. (2005) Hum. Mutat. 25(3): 322; Landen et al. (2008) J. Clin. Oncol. 26(6): 995-1005; Ramus et al. (2015) j Natl Cancer Inst 107(11).

In some embodiments of any of the methods described herein, the additional therapeutic intervention is chemotherapy (e.g., any of the platinum-based chemotherapeutic agents described herein (e.g., cisplatin, carboplatin), or a taxane (e.g., placitaxel (Taxol®) or docetaxel (Taxotere®). In some embodiments, the chemotherapeutic agent is an albumin-bound paclitaxel (nap-paclitaxel, Abraxane®), altretamine (Hexalen®), capecitabine (Xeloda®), cyclophosphamide (Cytoxan®), etoposide (VP-16), gemcitabine (Gemzar®), ifosfamide (Ifex®), irinotecan (CPT-11, Camptosar®), liposomal doxorubicin (Doxil®), melphalan, pemetrexed (Alimta®), topotecan, or vinorelbine (Navelbine®). In some embodiments, the therapeutic intervention is a combination of chemotherapeutic agents (e.g., paclitaxel, ifosfamide, and cisplatin; vinblastine, ifosfamide and cisplatin; etoposide, ifosfamide and cisplatin).

In some embodiments, the therapeutic intervention is an epigenetic therapy (see, e.g., Smith et al. (2017) Gynecol. Oncol. Rep. 20: 81-86). In some embodiments, the epigenetic therapy is a DNA methyltransferase (DNMT) inhibitor (e.g., 5-azacytidine (5-AZA), decitabine (5-aza-2′-deoxycytidine) (Fu et al. (2011) Cancer 117(8): 1661-1669; Falchook et al. (2013) Investig. New Drugs 31(5): 1192-1200; Matei et al. (2012) Cancer Res. 72(9): 2197-2205). In some embodiments, the DNMT1 inhibitor is NY-ESO-1 (Odunsi et al. (2014) Cancer Immunol. Res. 2(1): 37-49). In some embodiments, the epigenetic therapy is a histone deacetylase (HDAC) inhibitor. In some embodiments, the HDAC inhibitor is vorinostat (Modesitt (2008) 109(2): 182-186) or belinostat (Mackay et al. (2010) Eur. J. Cancer 46(9): 1573-1579). In some embodiments, the HDAC inhibitor is given in combination with a chemotherapeutic agent (e.g., carboplatin (paraplatin), cisplatin, paclitaxel or docetaxel (taxotere)) (Mendivil (2013) Int. J. Gynecol. Cancer 23(3): 533-539; Dizon (2012) Gynecol. Oncol. 125(2): 367-371; Dizon (2012) Int J. Gynecol. Cancer 23(3): 533-539).

In some embodiments, the therapeutic intervention is an anti-angiogenic agent (e.g., bevacizumab).

In some embodiments, the therapeutic intervention is a poly (ADP-ribose) polymerase (PARP)-1 and/or PARP-2 inhibitor. In some embodiments, the PARP-1 and PARP-2 inhibitor is niraparib (zejula) (Scott (2017) Drugs doiL10.1007/s40265-017-0752). In some embodiments, the PARP inhibitor is olaparib (lynparza) or rucaparib (rubraca).

In some embodiments, the therapeutic intervention is a hormone (e.g., a luteinizing-hormone-releasing hormone (LHRH) agonist). In some embodiments, the LHRH agonist is goserelin (Zoladex®) or leuprolide (Lupron®). In some embodiments, the therapeutic intervention is an anti-estrogen compound (e.g., tamoxifen). In some embodiments, the therapeutic intervention is an aromatase inhibitor (e.g., letrozole (Femara®), anastrozole (Arimidex®) or exemestane (Aromasin®).

In some embodiments, the therapeutic intervention is surgery (e.g., debulking of the tumor mass, a hysterectomy, a bilateral salpingo-oophorectomy, an omentectomy). The term “debulking” refers to surgical removal of almost the entire tumor (“optimally debulked”). In some embodiments, debulking can include removing a portion of the bladder, the spleen, the gallbladder, the stomach, the liver, and/or pancreas. In some embodiments, adjuvant chemotherapy is further administered to the subject after surgery (e.g., debulking of the tumor mass, a hysterectomy, a bilateral salpingo-oophorectomy, an omentectomy). In some embodiments, adjuvant chemotherapy is administered intra-abdominally (intraperitoneally). In some embodiments, the therapeutic intervention is a prophylactic surgery (e.g., a hysterectomy). In some embodiments, a paracentesis is performed to remove ascites.

In some embodiments, the therapeutic intervention is radiation therapy. In some embodiments, the radiation therapy is external beam radiation therapy, brachytherapy or a use of radioactive phosphorus.

### Lung Cancer

In some embodiments of any of the methods described herein, the subject may have hereditary lung cancer (Gazdar et al. (2014) J. Thorac. Oncol. 9(4): 456-63). In some embodiments, the subject has non-small cell-lung cancer (NSCLC) or small cell lung cancer (SCLC).

In some embodiments, the subject's lung cancer was caused by a somatic mutation in a gene. In some embodiments, the subject has a mutation in a gene selected from the group consisting of: ARIDIA, AKT anaplastic lymphoma kinase (ALK), BRAF, cyclin dependent kinase inhibitor 2 (CDKN2A), discoidin domain receptor tyrosine kinase 2 (DDR2), epidermal growth factor receptor (EGFR), fibroblast growth factor receptor 1 (FGFR1), HER2/ERBB2, kelch like ECH associated protein 1 (KEAP1) (Singh et al. (2006) PLoS Med 3: e420), KRAS proto-oncogene (KRAS), MAP kinase/ERK kinase 1 (MEK1), MET proto-oncogene (MET), MAX gene associated (MGA), myelocytomatosis oncogene (MYC), NF1, NRAS, neutrophophic receptor tyrosine kinase 1 (NTRK1), PTEN, PIK3CA, RB1, RNA binding motif protein 10 (RBM10), ret proto-oncogene (RET), Ras like without CAAX 1 (RIT1) (Berger et al. (2014) Oncogene), Ros proto-oncogene (ROSI), STE domain containing 2 (SETD2), SWI/SNF related matrix associated actin dependent regulator of chromatin, subfamily A, member 4 (SMARCA4) (Medina et al. (2008) Hum. Mutat. 29: 617-622), (SOX2) (Rudin et al. (2012) Nature Genet. 44: 111-1116), LKB1 (STKI1) (Sanchez-Cespedees et al. (2002) Cancer Res. 62: 3659-3662), TP53 (Takahashi et al. (1989) Science 246: 491-494), U2 small nuclear RNA auxillary factor 1 (U2AF1), and combinations thereof. See e.g., The Cancer Genome Atlas Research Network (2014) Nature 511: 543-550; Ding et al. (2008) Nature 1069-1075; The Cancer Genome Atlas Research Network (2012) Nature 489: 519-525; Seo et al. (2012) Genome Res. 22: 2109-2119; El-Telbany and Ma (2012) Genes Cancer 3(7-8): 467-480; Marks et al. (2008) Cancer Res. 68: 5524-5528; De Braud et al. (2014) J. Clin. Oncol. 32: 2502; Rothschild (2015) Cancers 7: 930949.

In some embodiments, a copy number variation or an oncogenic chromosomal gene rearrangement (e.g., oncogenic chromosomal translocation) is detected in a lung cancer cell. Non-limiting examples of oncogenic chromosomal translocation found in lung cancer include: EML4-ALK, TFG-ALK, KIF5B-ALK, KLC1-ALK, PTPN3-ALK, TPR-ALK, HIP1-ALK, STRN-ALK, DCTN1-ALK, SQSTM1-ALK, BIRC6-ALK, RET-PTC1, KIF4B-RET, CCDC6-RET and NCOA4-RET. See, e.g., Iyevleva et al. (2015) Cancer Lett. 362(1): 116-121; Wang et al. (2012) J. Clin Oncol. 30: 4352-9 In some embodiments, the therapeutic intervention is an anti-angiogenic agent (e.g., bevacizumab (avastin), ramucirumab (cyramza)).

In some embodiments, the therapeutic intervention is a targeted drug therapy. In some embodiments, the targeted drug therapy is an EGFR inhibitor (e.g., erlotinib (tarceva), afatinib (gilotrif), gefitinib (iressa), necitumumab (portrazza), cetuximab, osimertinib (AZD9291, Tagrisso), rociletinib (CO-1686), HM61713 (BI 1482694), ASP8273, EGF816, PF-06747775). See, e.g., Wang et al. (2016) J. Hematol Oncol 9:34; Cross et al. (2014) Cancer Discov. 4(9): 1046-61; Walter et al. (2013) Cancer Discov 3(12): 1404-15; Park et al. (2015) ASCO Meeting Abstract 33(15): 8084; Sequist et al. (2015) 372(18): 1700-9; Lee et al. (2014) Cancer Res 74(19Supplement):LB-100; Sakagami et al. (2014) Cancer Res 74(19 Supplement): 1728; Goto et al. (2015) ASCO Meeting Abstract 33(15_Suppl):8014; Jia et al. (2016) Cancer Res 76: 1591-602.

In some embodiments, the targeted drug therapy is an ALK inhibitor (e.g., crizotinib (xalkori), ceritinib (zykadia, LDK378), alectinib (alecensa, RO5424802; CH5424802), brigatinib (alunbrig, AP26113), lorlatinib (PF-06463922), TSR-011, RXDX-101 (NMS-E628), X-396, CEP-37440). See, e.g., Tartarone et al. (2017) Med. Oncol. 34(6): 110; Galkin et al. (2007) PNAS 104(1): 270-275; Friboulet et al. (2014) Cancer Discov. 4(6); 662-73; Chen et al. (2013) 56(14): 5673-5674; Shaw et al. (2014) N. Engl. J. Med. 370(13): 1189-1197; Sakamoto et al. (2011) Cancer Cell 19(5): 679-690; Squillace et al. (2013) Cancer Res. 73(8_suppl_: 5655; Mori et al. (2014) 13(2): 329-340; Patnaik et al. (2013) J. Clin. Oncol. 31 (15 suppl); Weiss et al. (2013) J Thorac Oncol. 8(suppl2): S618; Ardini et al. (2009) Mol. Cancer Ther. 8(12suppl): A244; Horn et al. (2014) J. Clin. Oncol. 32(15suppl); Cheng et al. (2012) Mol. Cancer Ther. 11(3): 670-679; Zhang et al. (2011) Cancer Res. 70(8suppl): LB-298; Awad and Shaw (2014) Clin. Adv. Hematol. Oncol. 12(7): 429-439.

In some embodiments, the targeted drug therapy is a heat shock protein 90 inhibitor (e.g, AUY922, ganetspib, AT13387). See, e.g., Pillai et al. (2014) Curr Opin Oncol. 26(2): 159-164; Normant et al. (2011) Oncogene 30(22): 2581-2586; Sequist et al. (2010) J. Clin. Oncol. 28(33): 4953-4960; Sang et al. (2013) Cancer Discov. 3(4): 430-443; Felip et al. (2012) Ann Oncol 23(suppl9); Miyajima et al. (2013) Cancer Res. 73(23): 7022-7033.

In some embodiments, the targeted drug therapy is a RET inhibitor (e.g., cabozantinib (XL184), vandetanib, alectinib, sorafenib, sunitinib, ponatinib) See, e.g., Drilon et al. (2013) Cancer Discov 3:6305; Gautschi et al. (2013) J. Thorac Oncol 8: e43-4; Kodama et al. (2014) Mol. Cancer Ther. 13: 2910-8; Lin et al. (2016) J. Thoracic Oncol. 11(11): 2027-2032; Rosell and Karachaliou (2016) Lancet 17(12): 1623-1625; Falchook et al. (2016) J. Clin Oncol. 34(15): e141-144; Shaw et al. (2013) Nat Rev Cancer 13: 772-787; Gozgit et al. (2013) Cancer Res 73 (Suppl. 1): 2084.

In some embodiments, the targeted drug therapy is a BRAF inhibitor (e.g., dabrafenib, vemurafenib). See, e.g., Planchard et al. (2013) J. Clin. Oncol. 31:8009; Gautschi et al. (2013) Lung Cancer 82: 365-367; Schmid et al. (2015) Lung Cancer 87: 85-87.

In some embodiments, the targeted drug therapy is a MET inhibitor (e.g., onartuzumab, ficlatuzumab, rilotumumab, tivantinib, crizotinib). See, e.g., Spigel et al. (2014) J. Clin. Oncol. 32: 8000; Patnail et al. (2014) Br. J. Cancer 111: 272-280; Gordon et al. (2010): Clin. Cancer Res. 16: 699-710; Sequist et al. (2011) J. Clin. Oncol. 29: 3307-3315; Zou et al. (2007) Cancer Res. 67: 4408-4417; Ou et al. (2011) J. Thorac. Oncol. 6: 942-946.

In some embodiments, the therapeutic intervention is administration of an immunotherapy. See, e.g., Smasundaram and Burns (2017) J. Hematol. Oncol. 10:87. In some embodiments, the immunotherapy is an anti-PD-1 agent (e.g., nivolumab) (Brahmer et al. (2012) N. Engl. J. Med. 366(26): 2455-2465; Gettinger et al. (2016) J. Clin. Oncol. 34(25)), pembrolizumab (Keytruda) (Garon et al. (2015) N. Engl. J. Med. 372(21): 2018-2028), durvalumab), nivolumab (opdivo)). In some embodiments, the immunotherapy is an anti-PD-L1 agent (e.g., atezolizumab (Fehrenbacher et al. (2016) Lancet 387(10030): 1837-1846, Rittmeyer et al. (2017) Lancet 389(10066): 255-265); atezolizumab (Tecentriq)). In some embodiments, the immunotherapy is an anti-CTLA-4 agent (e.g., ipilimumab or tremlimumab). In some embodiments, the immunotherapy is a combination therapy of an anti-PD-1 agent and an anti-CTLA-4 agent (e.g., nivolumab and ipilimumab (Herbset et al. (2015) 21(7): 1514-1524), pembrolizumab and ipilimumab (Gubens et al. (2016) ASCO Meeting Abstracts 34(15_suppl):9027), durvalumab and tremlimumab (NCT02542293. Study of 1st line therapy study of MEDI4736 with tremelimumab versus SoC in non-small-cell lung cancer (NSCLC) (NEPTUNE)).

In some embodiments, the immunotherapy is given in combination with a chemotherapeutic agent (e.g., Rizvi et al. (2016) J. Clin. Oncol. 34(25): 2969-79; Hall et al. (2016) ASCO Meeting Abstracts. 34(15_suppl):TPS9104).

In some embodiments, the therapeutic intervention is chemotherapy (e.g., cisplatin, carboplatin, paclitaxel, albumin-bound paclitaxel, docetaxel, gemcitabine, vinorelbine, irinotecan, etoposide, vinblastine or pemetrexed (alimta)). In some embodiments, the therapeutic intervention is a combination of at least two chemotherapeutic agents.

In some embodiments, the therapeutic intervention is surgery (e.g., a wedge resection (i.e. removal of a small section of diseased lung and a margin of healthy tissue); a segmental resection (segmentectomy) (i.e. removal of a larger portion of lung, but not an entire lobe); a lobectomy (i.e. removal of an entire lobe of one lung); a pneumonectomy (i.e. removal of an entire lung)), or a sleeve resection. The extent of surgical removal will depend on the stage of lung cancer and overall prognosis. In some embodiments, surgery is carried out by video-assisted thoracic surgery (VATS). In some embodiments, the therapeutic intervention is radiofrequency ablation (RFA).

In some embodiments, the therapeutic intervention is radiation therapy. In some embodiments, the radiation therapy is external beam radiation therapy (e.g., three-dimensional conformal radiation therapy (3D-CRT), intensity modulated radiation therapy (IMRT), stereotactic body radiation therapy (SBRT), brachytherapy or a use of radioactive phosphorus.

In some embodiments, the therapeutic intervention further comprises palliative care. In some embodiments, palliative care includes removal of pleural effusion by thoracentesis, pleurodesis or catheter placement. In some embodiments, palliative care includes removal of pericardial effusion by pericardiocentesis, a pericardial window. In some embodiments, the therapeutic intervention is photodynamic therapy (PDT), laser therapy or stent placement.

### Breast Cancer

In some embodiments of any of the methods described herein, the subject may have hereditary breast cancer (Peters et al. (2017) Gynecol Oncol pii: S0090-8258(17)30794-1). In some embodiments, the subject may have triple negative breast cancer (estrogen receptor negative, progesterone receptor negative and HE2-negative), hormone receptor positive (estrogen and/or progesterone receptor positive) breast cancer, hormone receptor negative (estrogen and/or progesterone receptor negative) breast cancer, HER2 positive breast cancer, HER2 negative breast cancer, inflammatory breast cancer or metastatic breast cancer.

In some embodiments wherein a breast cancer cell has been detected in the subject, the subject has at least one mutation in a gene selected from the group consisting of: BRCA1, BRCA2, ATM, CHD1, CHEK2, PALB2, STKI1, TP53, HER2 (ERBB2), CDK416, AKTI, GATA binding protein 3 (GATA3), RB1, lysine methyltransferase 2C (MLL3), mitogen-activated protein kinase 1 (MAP3K1), CDKN1B, T-box3(TBX3), runt related transcription factor 1 (RUNX1), core binding factor beta (CBFB), phosphoinositide-3-kinase regulatory subunit 1 (PIK3R1), protein tyrosine phosphatase non-receptor type 22 (PTPN22), protein tyrosine phosphatase receptor type D (PTPRD), NF1, splicing factor 3b subunit 1 (SF3B1), cyclin D3 (CCND3), T-box 5 (TBX5), CCCTC-binding factor (CTCF), forkhead box A1 (FOX1), PI3KCA, PTEN, mitogen-activated protein kinase 4 (MAP2K4), and combinations thereof. See, e.g., Nik-Zainal et al. (2016) Nature 534: 47-54; Bergamaschi et al. (2008) J. Pathol. 214: 357-367; Pleasance et al. (2010) Nature 463: 191-196; The Cancer Genome Atlas Network (2012) Nature 490:61-70; Usary et al. (2004) Oncogene 23: 7669-7678; Bachman et al. (2004) Cancer Biol. Ther. 3: 772-775; Saal et al. (2008) Nature Genet 40: 102-107; Troester et al. (2006) BMC Cancer 6: 276; Chandriani et al. (2009) PLoS One 4: e6693; Matsuda et al. (2017) Breast Cancer Res Treat 163(2): 263-272.

In some embodiments, the targeted drug therapy is a HER2 inhibitor (e.g., trastuzumab (Herceptin), pertuzumab (perjeta); ado-trastuzumab emtansine (T-DM1; Kadcyla); lapatinib (Tykerb), neratinib). See, e.g., Baselga et al. (2012) N Engl J Med 366: 109-119; Konecny et al. (2006) Cancer Res 66: 1630-1639, Xia et al. (2007) Cancer Res. 67: 1170-1175; Gomez et al. (2008) J Clin Oncol 26: 2999-30005; Wong et al. (2009) Clin. Cancer Res. 15: 2552-2558; Agus et al. (2002) Cancer Cell 2: 127-137; Lewis Philips et al. (2008) Cancer Res 68: 9280-9290.

In some embodiments, the targeted drug therapy is a cyclin-dependent kinase inhibitor (e.g., a CDK4/6 inhibitor (e.g., palbociclib (Ibrance®), ribociclin (Kisqali®), abemaciclib) (Turner et al. (2015) N Engl J Med 373: 209-219; Finn et al. (2016) N Eng J Med 375: 1925-1936; Ehab and Elbaz (2016) Breast Cancer 8: 83-91; Xu et al. (2017) J Hematol. Oncol. 10(1): 97; Corona et al. (2017) Cri Rev Oncol Hematol 112: 208-214; Barroso-Sousa et al. (2016) Breast Care 11(3): 167-173)).

In some embodiments, the targeted drug therapy is a PARP inhibitor (e.g., olaparib (AZD2281), veliparib (ABT-888), niraparib (MK-4827), talazoparib (BMN-673), rucaparib (AG-14699), CEP-9722) See, e.g., Audeh et al. (2010) Lancet 376: 245-251; Fong et al. (2009) N Engl J Med 361: 123-134; Livrahi and Garber (2015) BMC Medicine 13: 188; Kaufamn et al. (2015) J Clin. Oncol. 33: 244-250; Gelmon et al. (2011) Lancet Oncol. 12: 852-61; Isakoff et al. (2011) Cancer Res 71:P3-16-05; Sandhu et al. (2013) Lancet Oncol 14:882-92; Tutt et al. (2010) Lancet 376: 235-44; Somlo et al. (2013) J. Clin. Oncol. 31: 1024; Shen et al. (2013) CLin. Cancer Res. 19(18): 5003-15; Awada et al. (2016) Anticancer Drugs 27(4): 342-8.

In some embodiments, the targeted drug therapy is a mTOR inhibitor (e.g., everolimus (afinitor)). See, e.g., Gong et al. (2017) Oncotarget doi: 10.18632/oncotarget.16336; Louseberg et al. (2017) Breast Cancer 10: 239-252; Hare and Harvey (2017) Am J Cancer Res 7(3): 383-404.

In some embodiments, the targeted drug therapy is a heat shock protein 90 inhibitor (e.g., tanespimycin) (Modi et al. (2008) J. Clin Oncol. 26: s1027; Miller et al. (2007) J. Clin. Oncol. 25:s1115; Schulz et al. (2012) J Exp Med 209(2): 275-89).

In some embodiments, the targeted drug therapy further includes a bone-modifying drug (e.g., a bisphosphonate or denosumab (Xgeva)). See, e.g., Ethier et al. (2017) Curr Oncol Rep 19(3): 15; Abdel-Rahman (2016) Expert Rev Anticancer Ther 16(8): 885-91.

In some embodiments, the therapeutic intervention is a hormone (e.g., a luteinizing-hormone-releasing hormone (LHRH) agonist). In some embodiments, the LHRH agonist is goserelin (Zoladex®) or leuprolide (Lupron®). In some embodiments, the therapeutic intervention is an anti-estrogen compound (e.g., tamoxifen, fulvestrant (faslodex)). In some embodiments, the therapeutic intervention is an aromatase inhibitor (e.g., letrozole (Femara®), anastrozole (Arimidex®) or exemestane (Aromasin®).

In some embodiments, the therapeutic intervention is surgery (e.g., a lumpectomy, a single mastectomy, a double mastectomy, a total mastectomy, a modified radical mastectomy, a sentinel lymph node biopsy, an axillary lymph node dissection; breast-conserving surgery). The extent of surgical removal will depend on the stage of breast cancer and overall prognosis.

In some embodiments, the therapeutic intervention is radiation therapy. In some embodiments, the radiation therapy is partial breast irradiation or intensity-modulated radiation therapy.

In some embodiments, the therapeutic intervention is chemotherapy (e.g., capecitabine (xeloda), carboplatin (paraplatin), cisplatin (platinol), cyclophosphamide (neosar), docetaxel (docefrez, taxotere), doxorubicin (Adriamycin), pegylated liposomal doxorubicin (doxil), epirubicin (ellence), fluorouracil (5-FU, adrucil), gemcitabine (gemzar), methotrexate, paclitaxel (taxol), protein-bound paclitaxel (abraxane), vinorelbine (navelbine), eribulin (halaven), or ixabepilone (ixempra)). In some embodiments, the therapeutic intervention is a combination of at least two chemotherapeutic agents (e.g., doxorubicin and cyclophosphamide (AC); epirubicin and cyclophosphamide (EC); cyclophosphamide, doxorubicin and 5-FU (CAF); cyclophosphamide, epirubicin and 5-FU (CEF); cyclophosphamide, methotrexate and 5-FU (CMF); epirubicin and cyclophosphamide (EC); docetaxel, doxorubicin and cyclophosphamide (TAC); docetaxel and cyclophosphamide (TC).

Non-limiting aspects of these methods are described below, and can be used in any combination without limitation. Additional aspects of these methods are known in the art.

### EXAMPLES

The invention is further described in the following examples, which do not limit the scope of the claims.

### Example 1. Dynamics of Cell-Free Tumor Load

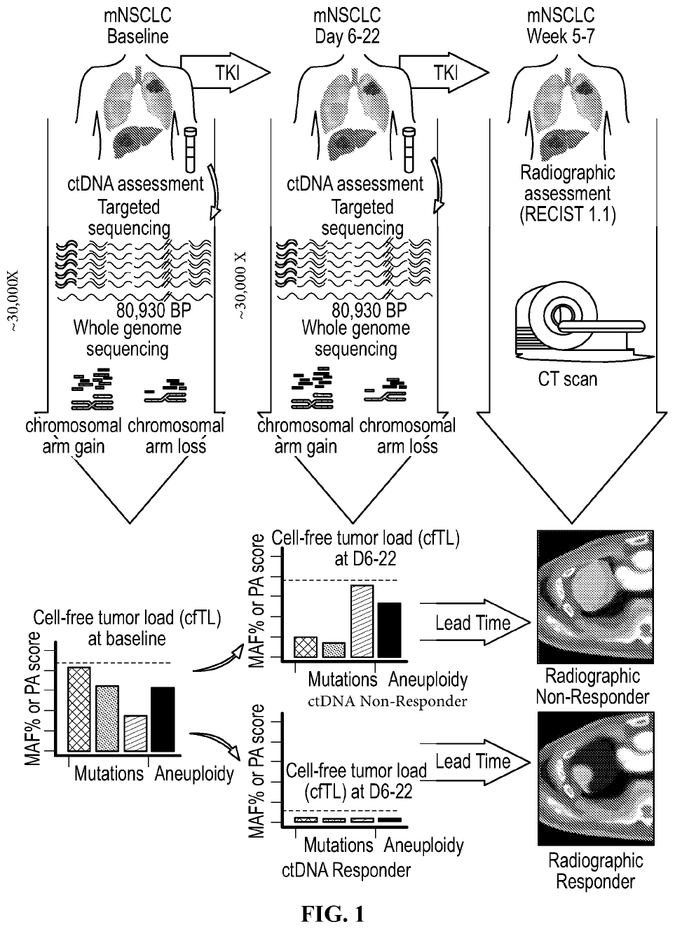

Serial blood was drawn from fifteen advanced non-small cell lung cancer (NSCLC) patients undergoing treatment with targeted tyrosine kinase inhibitors (TKIs), afatinib or osimertinib, to directly detect somatic sequence and structural alterations in cfDNA, to monitor ctDNA dynamics during therapy, to determine cell-free tumor burden, and to predict clinical outcome (FIG. 1). Liquid biopsies were obtained from 11 radiographic responders and four radiographic non-responders immediately prior to treatment (baseline), 6-22 days post treatment, and at serial time points until disease progression. The ultrasensitive targeted error correction sequencing (TEC-Seq) approach (6) was used as well as whole genome sequencing to identify tumor-derived sequence alterations and chromosomal copy number changes in cell-free DNA (cfDNA). The dynamics of alterations were identified, and developed a non-invasive measure of cfTL to evaluate real-time response to treatment. Next, changes in cfTL were evaluated within hours to days after treatment compared to baseline and assessed whether cfTL could serve as a marker of patient outcome.

For each patient, ˜5 ml of plasma were collected immediately prior to therapy (baseline), at 6-22 days after therapy initiation (median 16 days), and at additional serial time points until disease progression was confirmed by radiographic assessment (Tables S1 and S2). Target lesions were evaluated before therapy with CT/MRI imaging and approximately every six weeks until disease progression. Based on response assessment by RECIST 1.1 of the initial surveillance scans after therapy, nine of fifteen patients experienced partial response, one had stable disease, four progressed, and one patient had non-measurable disease due to bone-only metastases (Table S1). The patient with stable disease at the initial scan achieved a partial response on later scans.

To analyze changes in cfDNA in these patients and capture the clonal heterogeneity of metastatic disease, we developed a combined comprehensive approach for analysis of both sequence and chromosomal changes. For sequence analyses, the targeted error correction sequencing (TEC-Seq) approach was used to evaluate 58 well-known cancer driver genes (FIG. 1 and Tables S3, S4 and S5) (6). This method is based on targeted capture and deep sequencing (>30,000×) of DNA fragments to provide a high degree of specificity across 80,930 bp of coding gene regions and enables identification of tumor-specific alterations in ctDNA while distinguishing these from amplification and sequencing artifacts, germline changes, or alterations related to blood cell proliferation that may be present in cfDNA (6).

To evaluate chromosomal changes that may be present in ctDNA, whole genome sequences were used obtained from off-target fragments that were not captured during analysis of targeted regions in manner similar to other genome wide copy number analyses, including Digital Karyotyping and related NGS approaches (10, 26, 27). The most aberrant alterations in the genome representation of individual chromosome arms were used to construct a plasma aneuploidy score (PA-Score) that was evaluated to detect changes in ctDNA during therapy (Table S6).

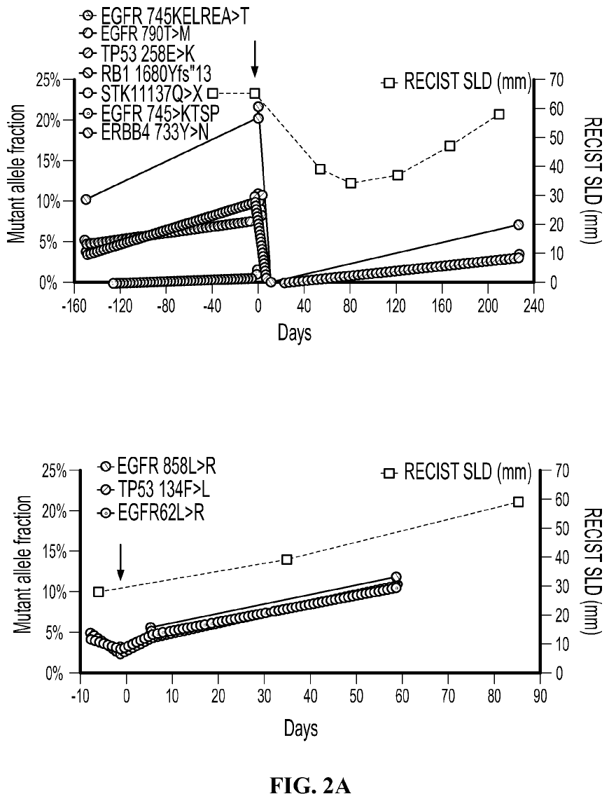

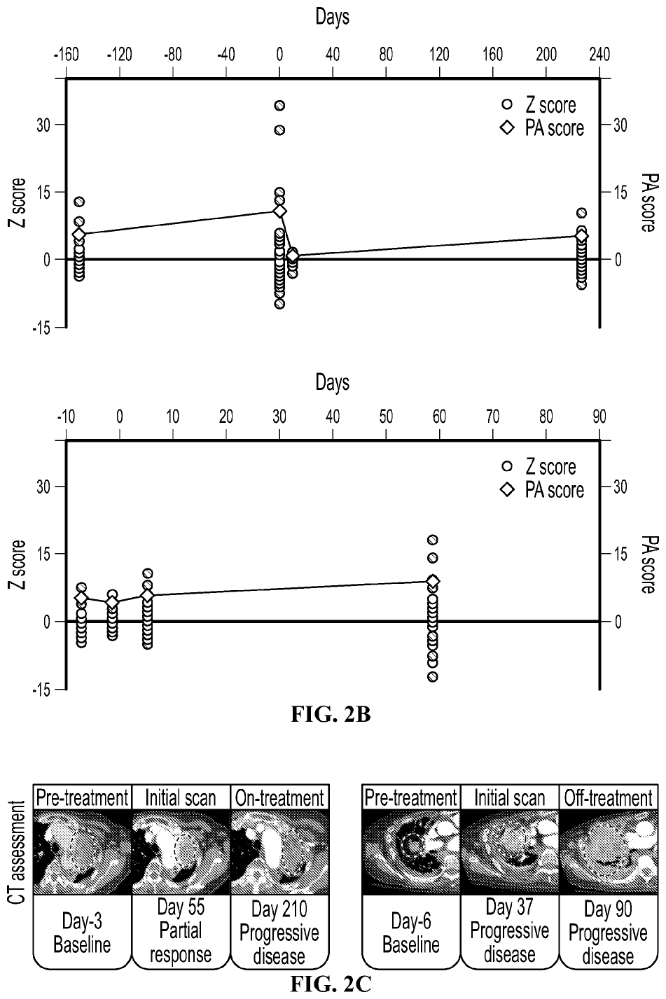

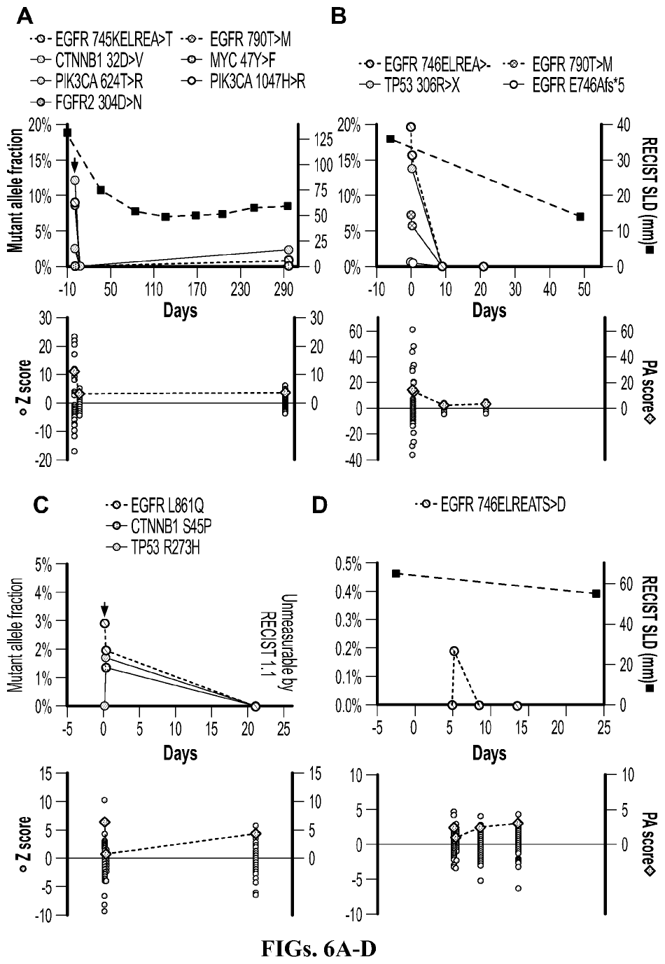

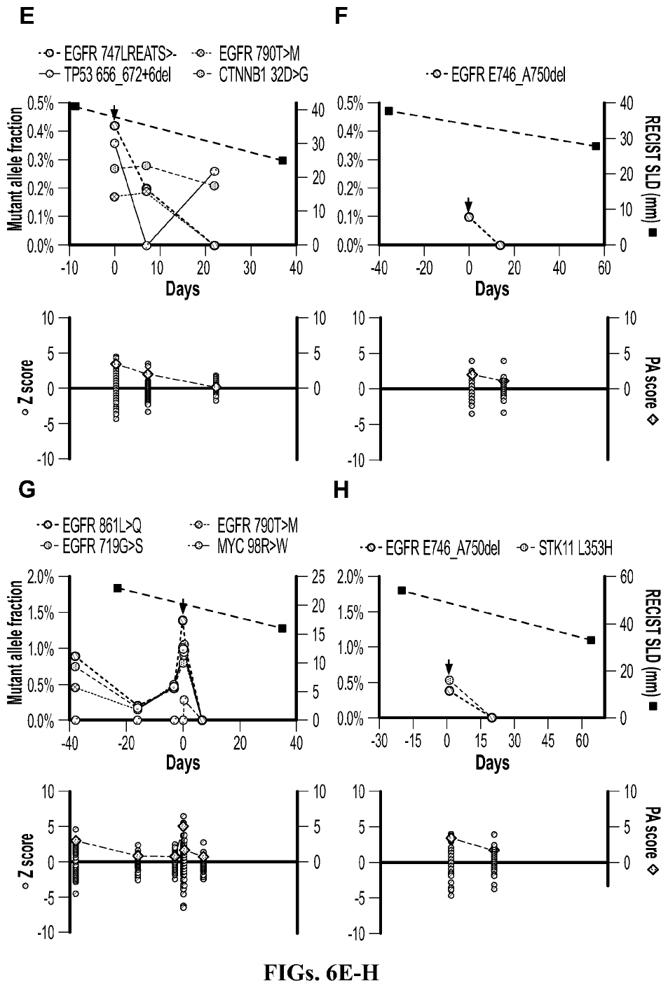

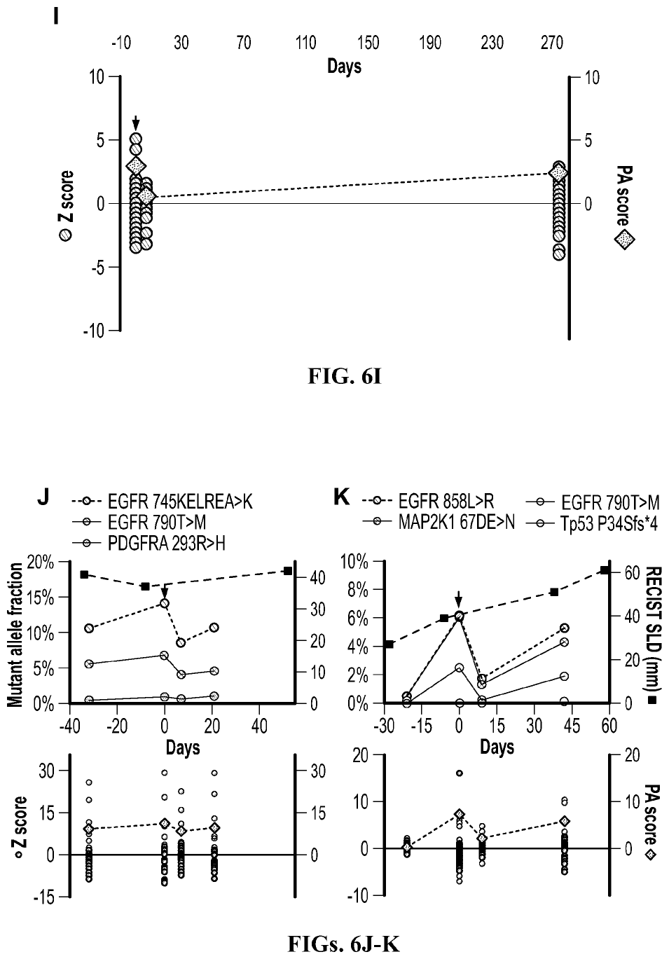

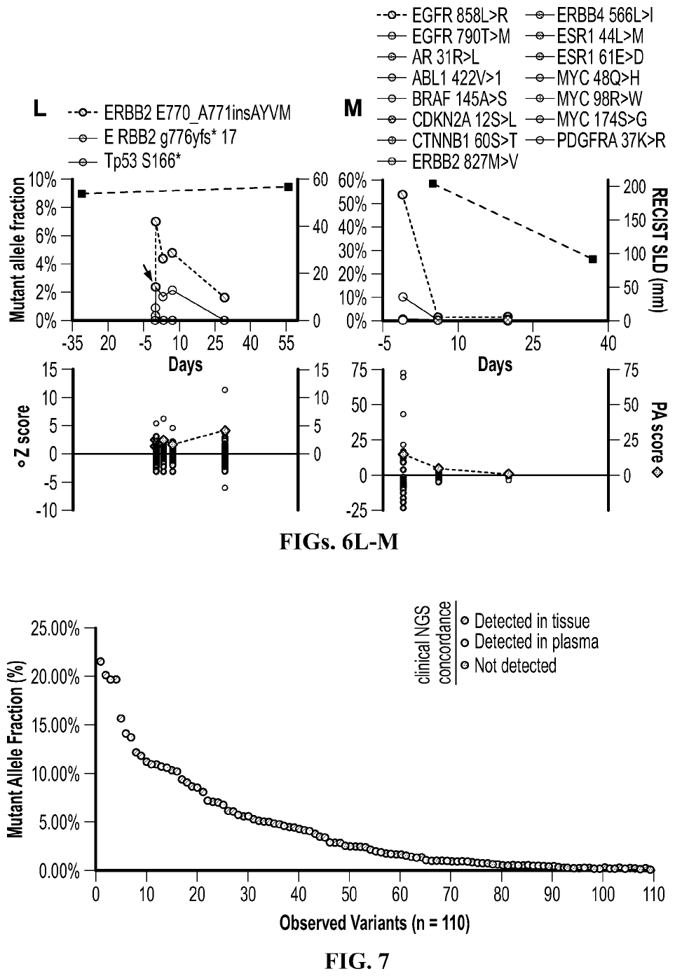

ctDNA was evaluated in all patients at baseline (pre-treatment) and 6-22 days after the initiation of therapy. In the blood draws that were analyzed, sequence alterations were detected in 14 of 15 cases. At the baseline time point, patients had an average of 3.6 tumor-specific somatic mutations, affecting 14 driver genes, ranging from one to 14 alterations per case (Table S5). At least one targetable mutation in either EGFR or ERBB2 was detected in each case analyzed, with ctDNA mutant allele fractions ranging from 0.10% to 53.71% (Table S5). Eight out of the 11 patients treated with osimertinib had EGFR T790M acquired resistance mutations in the circulation at baseline, with ctDNA mutant allele fractions ranging from 0.17% to 10.09% (Table S5), consistent with their previous treatment with EGFR TKIs (Table S1). Previously described alterations in genes involved in blood cell proliferation (28-32) were observed in nine patients across all time points analyzed, and were removed from further analyses (Table S7). Chromosomal abnormalities were detected in 13 of 15 cases. In most patients, multiple chromosomal arms were aberrant (FIG. 2, FIGS. 6A-M, Table S6), resulting in PA-Scores ranging from 1.3 to 14.9 at the baseline blood sample. Through the combined analyses, either a tumor-derived sequence or chromosomal change or both types of alterations were detected in all 15 cases.

Based on the alterations observed in cfDNA through analyses of multiple genes, a new metric, termed cell-free tumor load (cfTL), was developed, which is defined as the contribution of the most abundant alterations in cfDNA at any particular time point during the course of tumor evolution (FIG. 1 and Tables S5 and S6). In this study, the most abundant alterations were typically in driver genes targeted by the TKIs utilized (e.g., EGFR and ERBB2). A tiered approach to evaluate ctDNA levels was used, first using cfTL levels based on sequence changes, and then PA-Scores based on chromosomal changes if sequence alterations were not present. This approach had the benefit of providing a comprehensive assessment of tumor-derived alterations that would represent overall tumor burden during the course of disease and selective pressure of therapeutic interventions.

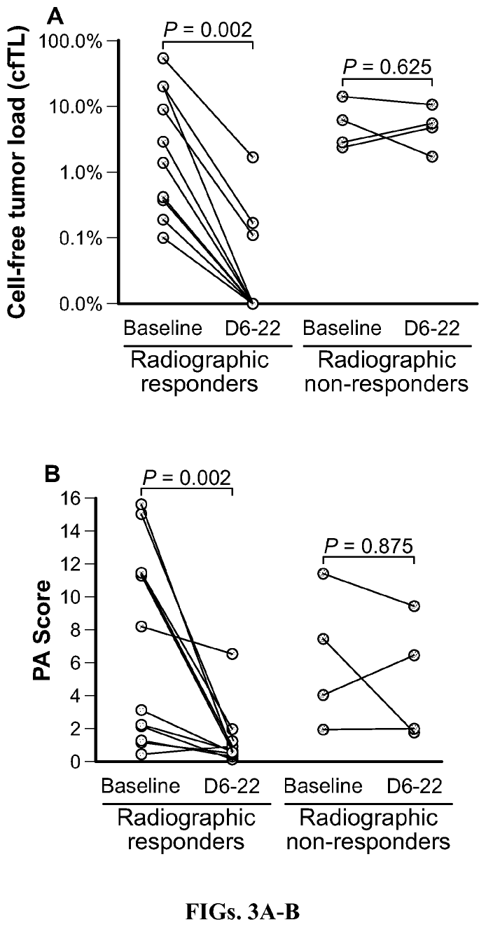

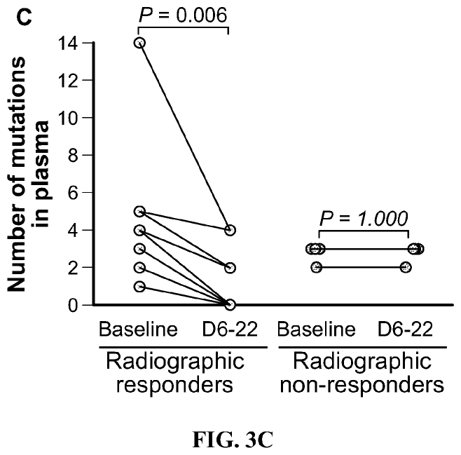

All patients with an initial objective radiographic response to targeted therapy displayed a rapid and dramatic reduction of cfTL (>95% decrease, P<0.01, Wilcoxon signed rank test) at 6-22 days after initiation of therapy (FIG. 2, FIGS. 6A-M and Table S). FIG. 2 depicts a representative patient with metastatic disease (CGPLLU2) who had a rapid decline of cfTL from baseline to day 10. This patient exhibited a progression-free survival of 7.0 months on osimertinib therapy, then subsequently developed resistance in the primary lung lesion. In patients with radiographic response or stable disease, mutant allele concentrations were reduced from an average of 10.80% at baseline to 0.20% at 6-22 days after treatment (>95% reduction) (FIG. 3A and Table S5) (P=0.002, Wilcoxon signed rank test). Likewise, PA-Scores decreased in responders (average decrease of 92%, P=0.002, Wilcoxon signed rank test), including in patient CGPLLU97 who had no sequence alterations detected in the plasma (FIG. 3B).

In contrast, all four patients who were radiographic non-responders to targeted therapy experienced limited variation in cfTL, as measured through both sequence and chromosomal alterations, ranging from an average mutant allele fraction of 6.38% at baseline to 5.70% at 6-22 days after treatment (FIGS. 2, 3A, 3B and FIGS. 6A-M) (P=0.6, Wilcoxon signed rank test).

In addition to changes in cfTL, the average number of observed alterations also decreased in responders from 4.0 to 1.2 mutations per patient (P=0.006, Wilcoxon signed rank test), while non-responders had no change in the number of mutations observed during therapy (2.5 mutations per patient both before and after therapy) (FIG. 3C).

Clinical NGS testing performed to identify alterations in tumor tissue or plasma during the care of these patients independently confirmed 86% of the changes detected in this study (FIG. 7). These observations suggested that both ctDNA levels and clonal heterogeneity were dramatically reduced at early time points in responding patients due to therapeutic selective pressure. In non-responding patients, the emergence and growth of tumor subclones could be detected earlier than radiographic progression.

### Example 2. Analysis of cfDNA within Hours of Therapy

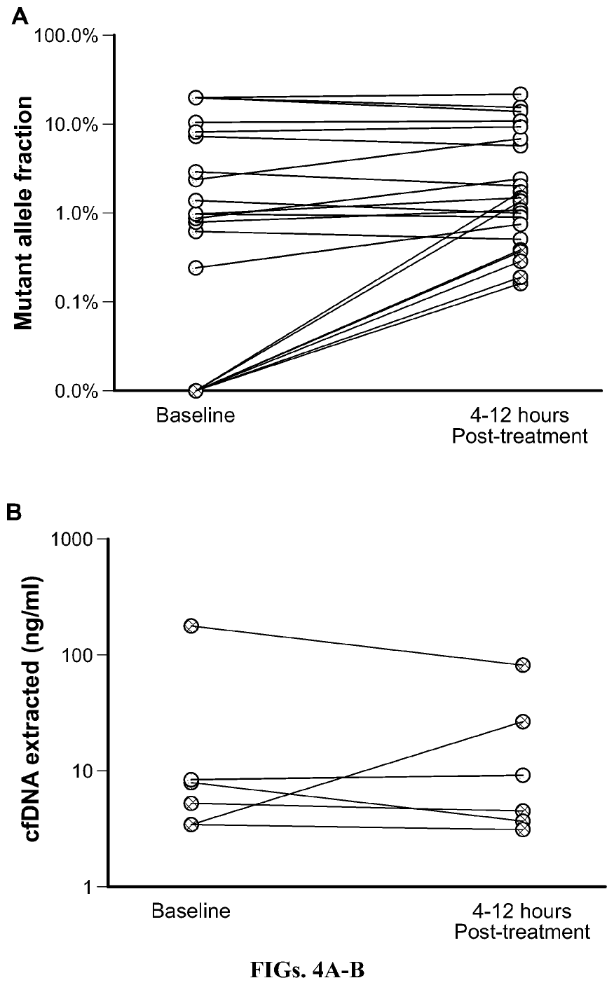

For a subset of patients, multiple follow-up blood draws were evaluated at extremely early time points in therapy. An immediate time point within the same day at 4-12 hours after the initiation of the first dose of treatment was available for four patients who experienced a partial radiographic response on the first or second scan (CGPLLU2, CGPLLU14, CGPLLU86, and CGPLLU99), one clinical responder classified with non-measurable disease by RECIST 1.1 (CGLU315), and one patient with progressive disease (CGLU294). In five of the six patients for whom immediate time points were evaluated, increasing ctDNA levels allowed for the identification of seven tumor-derived alterations not previously detected at baseline including the targetable EGFR 746ELREATS>D clone in patient CGPLLU86 detected at a mutant allele fraction of 0.19% (FIG. 4A). Mutant allele fractions of the newly detected clones ranged from 0.16% to 1.70% with an average of 0.63% and suggested that these alterations were likely below the limit of detection at baseline and were detected due to an increase in ctDNA levels. Remarkably, five of the six patients had newly emerging alterations within the same day after initiation of therapy compared to emerging alterations in two patients detected at earlier time points within 188 days prior to therapy. Evaluating the relative rate of emerging mutations in a Bayesian statistical model, we estimated a 110-fold increase in the rate of emerging mutations comparing post-treatment to pre-treatment levels (99% CI: 13-732). Overall cfDNA amounts remained relatively constant between baseline and time points 4-12 hours after treatment indicating that changes in ctDNA levels occurred due to changes in the relative abundance of mutated clones within cfDNA (FIG. 4B). These observations suggested that the emergence of novel ctDNA variants were related to early effects of therapy and are consistent with studies showing BIM-mediated apoptosis in responsive tumors 6-48 hours after exposure to EGFR inhibitors (33, 34).

### Example 3. Cell-Free Tumor Load and Clinical Outcome

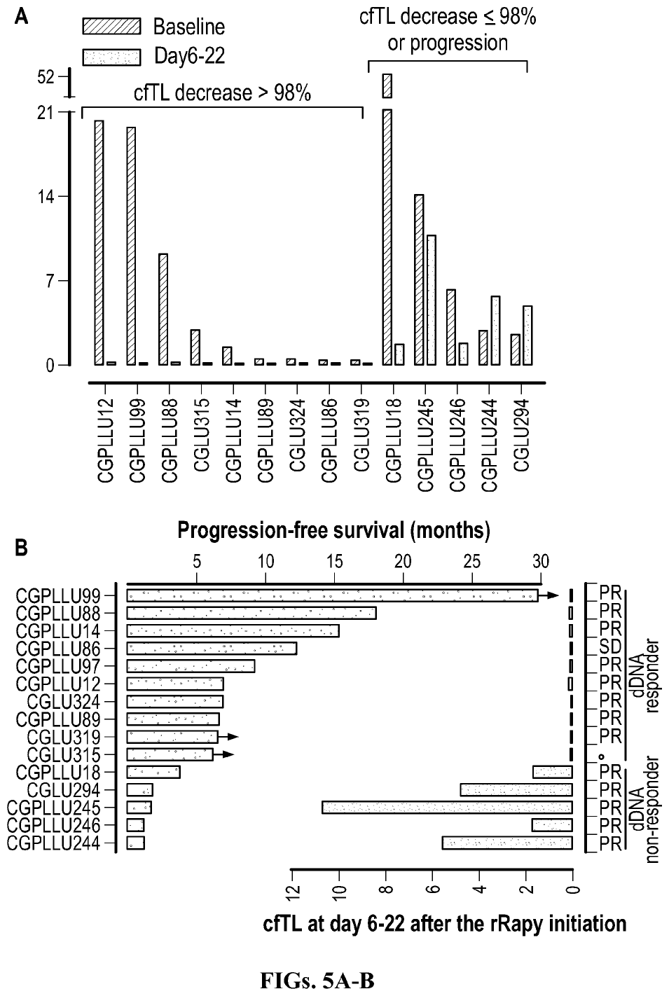

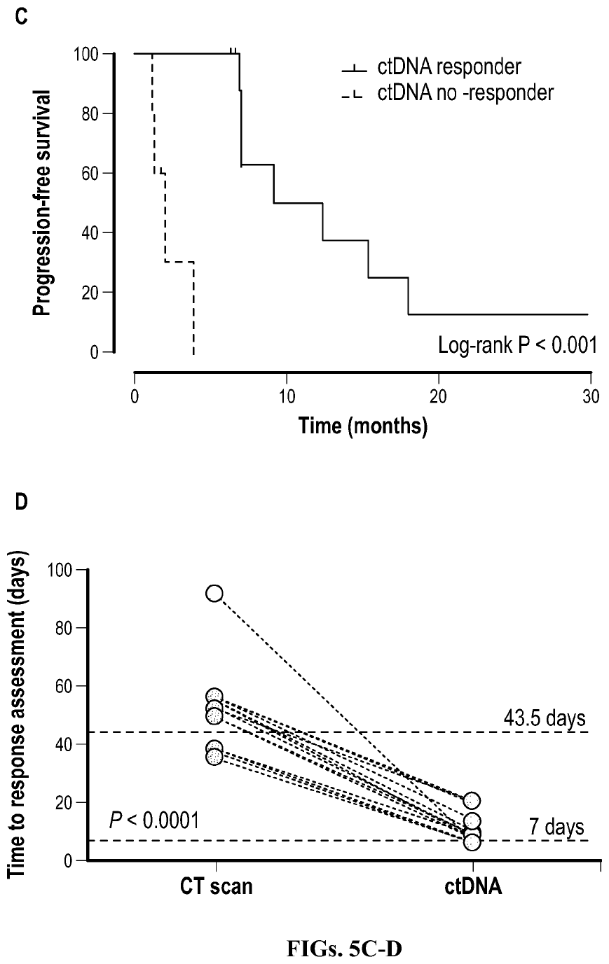

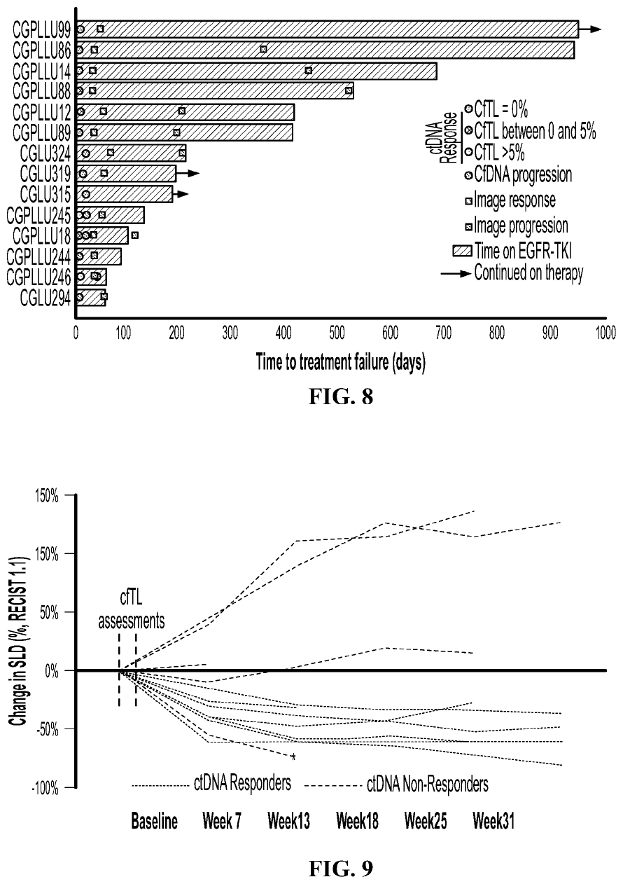

The dynamic cfTL changes observed at early time points after treatment initiation were associated with differences in clinical outcome. cfTL levels at day 6-22 were bimodal, with the lower group clustering at an average reduction in cfTL of 99.8% and the higher group having an average increase in cfTL of 0.8% (FIG. 5A). ctDNA responders were defined as those with reduction in cfTL levels within three standard deviations of average reduction of the lower group (greater than 98.7%) while non-responders were below this threshold. Three of eight patients who developed a complete ctDNA response (cfTL reduction of 100%) at day 6-22 experienced progression-free survival longer than one year, while two continue to respond, but have not yet reached one year of follow-up (FIG. 5B and FIG. 8).

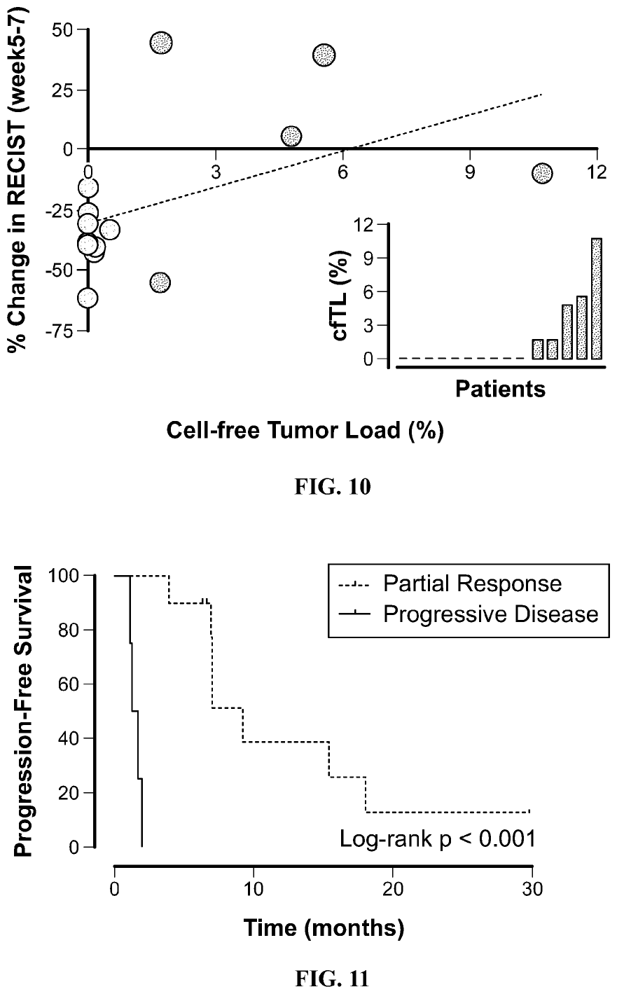

Three patients had different measures of response between ctDNA analyses at day 6-22 and initial scans after therapy (FIG. 5B, FIG. 9 and FIG. 10). For example, one of the ctDNA responders (CGPLLU86) with a cfTL reduction of 100% at day 7 and 17 post treatment who had a progression-free survival of 12.4 months was classified by RECIST as having stable disease at 38 days post treatment and achieved a maximal reduction of 46% only by the ninth CT scan (371 days) (FIG. 5B, FIG. 8 and FIG. 9). Similarly, another complete ctDNA responder (CGLU315) assessed at day 20 post treatment was classified as radiographically non-measurable yet continues to have a favorable clinical response and remains on treatment as of last follow-up (FIG. 5B FIG. 8). Although patient CGPLLU18 was classified by RECIST as a partial responder, cfTL analyses revealed that the patient was a ctDNA non-responder and had a limited therapeutic benefit (progression-free survival of 3.9 months) (FIG. 5B, FIG. 8 and FIG. 9).

Overall, we observed a significantly longer median progression-free survival for ctDNA responders at day 6-22 compared to ctDNA non-responders (10.8 months vs. 2.0 months, P<0.001, FIG. 5C). Importantly, cfTL reduction at day 6-22 was as accurate of a predictor of outcome as initial CT imaging performed an average of 48 days after initiation of therapy (FIG. 5C, FIG. 5D and FIG. 11.

Faster and predictive determinants of patient response can aid in navigating adaptive therapeutic strategies to reduce toxicity, identify early resistance to targeted therapy, and enable the consideration of combinatorial approaches early in a patient's therapy. Additionally, as occurred in patient CGPLLU86, these data indicate the utility of a single dose of treatment to elicit levels of ctDNA high enough to identify possible tumor-derived alterations.

### Example 4. Methods

### Patient and Sample Characteristics

Fifteen patients with metastatic non-small cell lung cancer undergoing treatment with tyrosine kinase inhibitors (TKIs) were included in our study. Clinical and pathological characteristics for all patients are summarized in Tables S1 and S2, and tumor load dynamics are shown in FIG. 2 and in FIGS. 6A-M. Patient enrollment and genomic studies were conducted in accordance with the Declaration of Helsinki, were approved by the Institutional Review Board (IRB) and patients provided written informed consent for sample acquisition for research purposes.

Of the fifteen patients, four were initially diagnosed with stage IIA or IIIA disease and treated with chemotherapy followed by osimertinib upon disease progression, and eleven were initially diagnosed with advanced disease and either treated with chemotherapy prior to osimertinib (n=2) or treated directly with first-line TKI therapy (n=9). Of the nine patients who received first-line TKI treatment, five received osimertinib and four received afatinib. Osimertinib was dosed at 80 mg PO daily and afatinib at 40 mg PO daily (Table S).

The response evaluation criteria in solid tumors (RECIST) version 1.1 (35) were used for assessment of response. Of the fifteen patients analyzed, nine achieved a partial response based on their initial CT assessment post treatment while one patient exhibited stable disease and one patient had unmeasurable disease. Upon follow-up of these eleven patients, eight eventually experienced disease progression while two continue to derive clinical benefit from targeted inhibition, one having continued response and one having unmeasurable disease. Four of fifteen patients had progressive disease evident on the initial CT assessment post treatment and did not exhibit radiographic response by RECIST 1.1 (Table S1).

For all patients, serial blood draws were collected over the course of treatment with targeted inhibition for isolation of plasma and extraction of cell-free DNA (cfDNA) for genomic analyses. Time points were analyzed immediately prior to treatment for baseline assessment as well as 6-22 days post treatment and at serial time points until disease progression (Table S2).

Sample Preparation and Next-Generation Sequencing of cfDNA

Whole blood was collected in K2 EDTA tubes or Streck tubes and processed immediately or within 2 hours after storage at 4° C. for EDTA tubes or room temperature for Streck tubes, respectively. Plasma and cellular components were separated by centrifugation at 800 g for 10 minutes at 4° C. Plasma was centrifuged a second time at 18,000 g at room temperature to remove any remaining cellular debris and stored at −80° C. until the time of DNA extraction. DNA was isolated from plasma using the QiAmp® Circulating Nucleic Acids Kit (Qiagen GmbH) and eluted in LoBind tubes (Eppendorf®AG). Concentration and quality of cfDNA was assessed using the Bioanalyzer 2100 (Agilent Technologies).

TEC-Seq next-generation sequencing cell-free DNA libraries were prepared from 11 to 350 ng of cfDNA. Genomic libraries were prepared as previously described (6). Briefly, the NEBNext® DNA Library Prep Kit for Illumina (New England Biolabs (NEB)) was used with four main modifications to the manufacturer's guidelines: i) The library purification steps utilized the on-bead Agencourt® Ampure® XP approach, ii) reagent volumes were adjusted accordingly to accommodate the on-bead strategy, iii) a pool of 8 unique Illumina dual index adapters with 8 bp barcodes were used in the ligation reaction, and iv) cfDNA libraries were amplified with Hotstart Phusion™ Polymerase. Genomic library preparation was performed as previously described (6). Concentration and quality of cfDNA genomic libraries were assessed using the Bioanalyzer 2100 (Agilent Technologies).

Targeted capture was performed using the Agilent SureSelect reagents and a custom set of hybridization probes targeting 58 genes (Table 1 and Table S3) per the manufacturer's guidelines. The captured library was amplified with HotStart Phusion™ Polymerase (NEB). The concentration and quality of captured cfDNA libraries was assessed on the Bioanalyzer (Agilent Technologies). TEC-seq libraries were sequenced using 100-bp paired end runs on the Illumina HiSeq 2500 (Illumina).

TABLE 1
Genes Analyzed

| Gene | Region Analyzed |
|---|---|
| ABL1 | Specific Exons |
| AKT1 | Specific Exons |
| ALK | Full Coding Region |
| APC | Specific Exons |
| AR | Full Coding Region |
| ATM | Specific Exons |
| BRAF | Full Coding Region |
| CDH1 | Specific Exons |
| CDK4 | Full Coding Region |
| CDK6 | Full Coding Region |
| CDKN2A | Specific Exons |
| CSF1R | Specific Exons |
| CTNNB1 | Specific Exons |
| DNMT3A | Specific Exons |
| EGFR | Full Coding Region |
| ERBB2 | Specific Exons |
| ERBB4 | Full Coding Region |
| ESR1 | Full Coding Region |
| EZH2 | Specific Exons |
| FBXW7 | Specific Exons |
| FGFR1 | Specific Exons |
| FGFR2 | Specific Exons |
| FGFR3 | Specific Exons |
| FLT3 | Specific Exons |
| GNA11 | Specific Exons |
| GNAQ | Specific Exons |
| GNAS | Specific Exons |
| HNF1A | Specific Exons |
| HRAS | Full Coding Region |
| IDH1 | Specific Exons |
| IDH2 | Specific Exons |
| JAK2 | Full Coding Region |
| JAK3 | Specific Exons |
| KDR | Specific Exons |
| KIT | Full Coding Region |
| KRAS | Full Coding Region |
| MAP2K1 | Specific Exons |
| MET | Specific Exons |
| MLH1 | Specific Exons |
| MPL | Specific Exons |
| MYC | Specific Exons |
| NPM1 | Specific Exons |
| NRAS | Full Coding Region |
| PDGFRA | Full Coding Region |
| PIK3CA | Full Coding Region |
| PIK3R1 | Specific Exons |
| PTEN | Full Coding Region |
| PTPN11 | Specific Exons |
| RB1 | Specific Exons |
| RET | Specific Exons |
| SMAD4 | Specific Exons |
| SMARCB1 | Specific Exons |
| SMO | Specific Exons |
| SRC | Specific Exons |
| STK11 | Full Coding Region |
| TERT | Specific Exons |
| TP53 | Full Coding Region |

### Primary Processing of Next-Generation Sequencing Data and Identification of Putative Somatic Mutations

Primary processing of next-generation sequence data for analyses of sequence alterations in cfDNA samples was performed as previously described (6). Briefly, Illumina CASAVA (Consensus Assessment of Sequence and Variation) software (version 1.8) was used for de-multiplexing and masking of dual index adapter sequences. Sequence reads were aligned against the human reference genome (hg19) using NovoAlign with additional realignment of select regions using the Needleman-Wunsch method (36).

Candidate somatic mutations, consisting of point mutations, small insertions, and deletions were identified using VariantDx (36) across the targeted regions of interest as previously described (6). Briefly, an alteration was considered a candidate somatic mutation only when: (i) Three distinct paired reads contained the mutation in the plasma and the number of distinct paired reads containing a particular mutation in the plasma was at least 0.1% of the total distinct read pairs; or (ii) Four distinct paired reads contained the mutation in the plasma and the number of distinct paired reads containing a particular mutation in the plasma was at least 0.05% and less than 0.1% of the total distinct read pairs; (iii) the mismatched base was not present in >1% of the reads in a panel of unmatched normal samples as well as not present in a custom database of common germline variants derived from dbSNP; (iv) the altered base did not arise from misplaced genome alignments including paralogous sequences; and (v) the mutation fell within a protein coding region and was classified as a missense, nonsense, frameshift, or splice site alteration.

Candidate alterations were defined as somatic hotspots if the nucleotide change and amino acid change were identical to an alteration observed in ≥20 cancer cases reported in the COSMIC database. Alterations that were not hotspots were retained only if either (i) seven or more distinct paired reads contained the mutation in the plasma and the number of distinct paired reads containing a particular mutation in the plasma was at least 0.1% and less than 0.2%, of the total distinct read pairs, or (ii) six or more distinct paired reads contained the mutation in the plasma and the number of distinct paired reads containing a particular mutation in the plasma was at least 0.2% of the total distinct read pairs. In order to track clonal changes over time, any alteration identified in at least one blood draw was followed in the remaining serial timepoints regardless of whether mutant allele fractions fit the criteria defined to call a single hotspot or non-hotspot mutation.

Common germline variants were identified and removed if present in ≥25% of reads or <25% of reads if the variant was recurrent and the majority of alterations at that position had a mutant allele fraction ≥25%. Variants known to be at a somatic hotspot position, or producing a truncating mutation in a tumor suppressor gene were not excluded as germline changes. Because of the high frequency of mutations in specific genes and the possible confounding between somatic and germline changes, we limited analyses in the APC gene to frameshift or nonsense mutations, and in KRAS, HRAS and NRAS to positions to 12, 13, 61, and 146. Finally, we excluded hematopoietic expansion related variants that have been previously described (28-32), including those in DNMT3A, IDH1, and IDH2 and specific alterations within ATM, GNAS, JAK2, or TP53 (Table S3 and Table S6).

Primary processing of next-generation sequence data for analyses of copy number alterations in cfDNA samples was performed as follows: Bam files were preprocessed by successively running CleanSam and MarkDuplicates from Picard Tools version 2.9.0. (http://broadinstitute.github.io/picard). Sequence reads were aligned against the human reference genome (hg19) using NovoAlign.

Candidate somatic structural variants were identified through analyses of low-coverage whole-genome sequencing data obtained from off-target reads mapping outside of the targeted capture of 58 cancer driver genes (Table 1 and Table S3) in areas of the genome farther than 1000 base pairs from the start or end of a targeted region. Off-target reads were divided into 100 kb bins with the exception of filtered bins i) with less than 10 kb due to spacing of target regions, ii) having GC content less than 30% or greater than 70%, iii) where 25% fell within the ENCODE Duke Excluded Regions Track (http://genome.ucsc.edu/cgi-bin/hgFileUi?db=hg19&g=wgEncodeMapability). The total number of unique reads mapping to each bin were counted to exclude filtered regions:

$${s}_{b} = {log}_{2} ( \frac{100000}{{x}_{b} - {f}_{b}} \cdot {r}_{b} )$$

where r_{b} is the number of unique reads mapped to bin b, x_{b} is the length of bin b, and fb is the number of filtered base pairs within bin b, and the normalized score, s_{b}, was assigned to each bin. To remove GC-bias and normalize for sequencing depth we used LOESS smoothing to predict a bin's normalized score from the bin-specific GC content. The GC-corrected score for each bin, [symbol] , is defined for bin b by subtracting the predicted score from s_{b} and exponentiating this using base 2. We summed the GC-corrected scores for each chromosome arm. The summed score for a given chromosome arm was divided by the summed score using all bins to calculate the percentage of genomic representation.

Z scores were calculated as previously described (10) for each chromosome arm for each time point and patient assessed to determine areas of genome over or under representation. PA scores were calculated as previously described (10) for each time point for each patient assessed in order to concisely represent the aneuploidy observed in each sample by using the five chromosomes arms with the largest absolute z scores. PA scores higher than the threshold score of 2.4 provide a specificity greater than 90% (Student t distribution, three degrees of freedom) for the presence of aneuploid circulating tumor DNA.

### Cell-Free Tumor Load

Sequence and copy number alterations were detected in cfDNA for each patient at each time point analyzed and used a tiered approach to evaluate tumor burden. For patients with detectable sequence alterations, the mutant allele fraction of the most abundant alteration in a clone targeted by the TKI was used as readout of cfTL. In patients without detectable sequence alterations, we evaluated the PA score as a binary readout of cfTL where a score above 2.4 indicated aneuploidy and evidence of tumor burden and a score below 2.4 indicated normal ploidy and the absence of detectable tumor burden in plasma.

Changes in cfTL were evaluated to compare tumor burden at baseline and at other time points during treatment using quantitative assessment of cfTL mutant allele fractions for patients with detectable sequence clones and qualitative assessment of change from aneuploidy to normal ploidy representing a complete response for patients without detectable sequence clones.

### Statistical Analysis

Significance was determined using a variety of methods. To assess the significance of reduction in cfTL (FIG. 3A), reduction in PA scores (FIG. 3B), and change in the number of sequence mutations detected (FIG. 3C) in patients with radiographic response or stable disease versus patients with no radiographic response post treatment we used the Wilcoxon signed rank test. The rates of emerging mutations in the presence (within 4-12 hours) and absence of selective pressure of therapy were compared using a Gamma-Poisson Bayesian model. A Gamma (1, 100) prior was used for both mutation rates. Reported rates were based on the posterior mean and 99% posterior credible intervals (CI) (FIG. 4). We compared progression-free survival in ctDNA responders versus ctDNA non-responders (FIG. 5C) as well as in RECIST responders versus RECIST non-responders (FIG. 9) using the Mantel-Cox log-rank test. Paired t test was used to assess the difference in the time to response assessment post therapy based on ctDNA analyses versus RECIST (FIG. 5D).

### REFERENCES

1. C. Sawyers, Targeted cancer therapy. Nature 432, 294 (Nov. 18, 2004).
2. B. Vogelstein et al., Cancer genome landscapes. Science 339, 1546 (Mar. 29, 2013).
3. D. A. Haber, V. E. Velculescu, Blood-based analyses of cancer: circulating tumor cells and circulating tumor DNA. Cancer discovery 4, 650 (June, 2014).
4. A. M. Newman et al., An ultrasensitive method for quantitating circulating tumor DNA with broad patient coverage. Nat Med 20, 548 (May, 2014).
5. C. Bettegowda et al., Detection of circulating tumor DNA in early- and late-stage human malignancies. Sci Transl Med 6, 224ra24 (Feb. 19, 2014).
6. J. Phallen et al., Direct detection of early-stage cancers using circulating tumor DNA. Sci Transl Med 9, (Aug. 16, 2017).
7. C. Alix-Panabieres, K. Pantel, Clinical Applications of Circulating Tumor Cells and Circulating Tumor DNA as Liquid Biopsy. Cancer discovery 6, 479 (May, 2016).
8. G. Siravegna, S. Marsoni, S. Siena, A. Bardelli, Integrating liquid biopsies into the management of cancer. Nature reviews. Clinical oncology 14, 531 (September, 2017).
9. F. Diehl et al., Circulating mutant DNA to assess tumor dynamics. Nat Med 14, 985 (September, 2008).
10. R. J. Leary et al., Detection of chromosomal alterations in the circulation of cancer patients with whole-genome sequencing. Sci Transl Med 4, 162ra154 (Nov. 28, 2012).
11. L. A. Diaz, Jr. et al., The molecular evolution of acquired resistance to targeted EGFR blockade in colorectal cancers. Nature 486, 537 (Jun. 28, 2012).
12. C. Rago et al., Serial Assessment of Human Tumor Burdens in Mice by the Analysis of Circulating DNA. Cancer Res 67, 9364 (Oct. 1, 2007).
13. J. Tie et al., Circulating tumor DNA as an early marker of therapeutic response in patients with metastatic colorectal cancer. Annals of oncology: official journal of the European Society for Medical Oncology/ESMO 26, 1715 (August, 2015).
14. R. J. Leary et al., Development of personalized tumor biomarkers using massively parallel sequencing. Sci Transl Med 2, 20ra14 (Feb. 24, 2010).
15. D. J. McBride et al., Use of cancer-specific genomic rearrangements to quantify disease burden in plasma from patients with solid tumors. Genes Chromosomes Cancer 49, 1062 (November, 2010).
16. F. Garlan et al., Early Evaluation of Circulating Tumor DNA as Marker of Therapeutic Efficacy in Metastatic Colorectal Cancer Patients (PLACOL Study). Clin Cancer Res 23, 5416 (Sep. 15, 2017).
17. M. Yanagita et al., A Prospective Evaluation of Circulating Tumor Cells and Cell-Free DNA in EGFR-Mutant Non-Small Cell Lung Cancer Patients Treated with Erlotinib on a Phase II Trial. Clin Cancer Res 22, 6010 (Dec. 15, 2016).
18. N. Pecuchet et al., Base-Position Error Rate Analysis of Next-Generation Sequencing Applied to Circulating Tumor DNA in Non-Small Cell Lung Cancer: A Prospective Study. PLoS Med 13, e1002199 (December, 2016).
19. S. J. Dawson et al., Analysis of circulating tumor DNA to monitor metastatic breast cancer. N Engl J Med 368, 1199 (Mar. 28, 2013).
20. C. Abbosh et al., Phylogenetic ctDNA analysis depicts early-stage lung cancer evolution.

Nature 545, 446 (Apr. 26, 2017).
21. C. P. Paweletz et al., Bias-Corrected Targeted Next-Generation Sequencing for Rapid, Multiplexed Detection of Actionable Alterations in Cell-Free DNA from Advanced Lung Cancer Patients. Clin Cancer Res 22, 915 (Feb. 15, 2016).
22. J. De Greve et al., Clinical activity of afatinib (BIBW 2992) in patients with lung adenocarcinoma with mutations in the kinase domain of HER2/neu. Lung cancer 76, 123 (April, 2012).
23. L. V. Sequist et al., Phase III study of afatinib or cisplatin plus pemetrexed in patients with metastatic lung adenocarcinoma with EGFR mutations. J Clin Oncol 31, 3327 (Sep. 20, 2013).
24. T. S. Mok et al., Osimertinib or Platinum-Pemetrexed in EGFR T790M-Positive Lung Cancer. N Engl J Med 376, 629 (Feb. 16, 2017).
25. P. A. Janne et al., AZD9291 in EGFR inhibitor-resistant non-small-cell lung cancer. N Engl J Med 372, 1689 (Apr. 30, 2015).
26. E. Heitzer et al., Tumor-associated copy number changes in the circulation of patients with prostate cancer identified through whole-genome sequencing. Genome medicine 5, 30 (2013).
27. T. L. Wang et al., Digital karyotyping. Proc Natl Acad Sci USA 99, 16156 (Dec. 10, 2002).
28. A. H. Shih et al., Mutational analysis of therapy-related myelodysplastic syndromes and acute myelogenous leukemia. Haematologica 98, 908 (June, 2013).
29. G. Genovese et al., Clonal hematopoiesis and blood-cancer risk inferred from blood DNA sequence. N Engl J Med 371, 2477 (Dec. 25, 2014).
30. S. Jaiswal et al., Age-related clonal hematopoiesis associated with adverse outcomes. N Engl J Med 371, 2488 (Dec. 25, 2014).
31. M. Xie et al., Age-related mutations associated with clonal hematopoietic expansion and malignancies. Nat Med 20, 1472 (December, 2014).
32. T. N. Wong et al., Role of TP53 mutations in the origin and evolution of therapy-related acute myeloid leukaemia. Nature 518, 552 (Feb. 26, 2015).
33. D. B. Costa et al., BIM mediates EGFR tyrosine kinase inhibitor-induced apoptosis in lung cancers with oncogenic EGFR mutations. PLoS Med 4, 1669 (October, 2007).
34. Y. Gong et al., Induction of BIM is essential for apoptosis triggered by EGFR kinase inhibitors in mutant EGFR-dependent lung adenocarcinomas. PLoS Med 4, e294 (Oct. 9, 2007).
35. L. H. Schwartz et al., RECIST 1.1-Update and clarification: From the RECIST committee. Eur J Cancer 62, 132 (July, 2016).
36. S. Jones et al., Personalized genomic analyses for cancer mutation discovery and interpretation. Sci Transl Med 7, 283ra53 (Apr. 15, 2015).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining the efficacy of a targeted therapy in a subject having cancer, comprising:
detecting a first cell-free tumor load (cfTL) in a biological sample isolated from the subject at a first time point;
detecting a second cfTL in a biological sample obtained from the subject at a second time point, wherein the subject has received at least one dose of the targeted therapy between the first time point and the second time point; and
identifying the targeted therapy as being effective in the subject when the subject exhibits a second cfTL that is reduced as compared to the first cfTL.

2. The method of claim 1, wherein:
detecting the first cfTL comprises detecting a first level of the at least one genetic alteration in circulating tumor DNA (ctDNA) in the biological sample isolated from the subject at the first time point, wherein the first cfTL corresponds to the first level of the at least one genetic alteration; and
detecting the second cfTL comprises detecting a second level of the at least one genetic alteration in circulating tumor DNA (ctDNA) in the biological sample isolated from the subject at the second time point, wherein the second cfTL corresponds to the second level of the at least one genetic alteration.

3. (canceled)

4. The method of claim 3, wherein detecting the first level of the at least one genetic alteration, detecting the second level of the at least one genetic alteration, or both comprises:
extracting cell-free DNA from blood;
ligating a low complexity pool of dual index barcode adapters to the cell-free DNA to generate a plurality of barcode adapter-ligated cell-free DNA segments;
capturing the plurality of barcode adapter-ligated cell-free DNA segments;
sequencing the plurality of captured barcode adapter-ligated cell-free DNA segments;
aligning the sequenced plurality of captured barcode adapter-ligated cell-free DNA segments to a reference genome; and
identifying sequence alterations using aligned sequences of multiple distinct molecules containing identical redundant changes.

5. The method of claim 2, wherein the at least one genetic is a mutation is in an EGFR gene, an ERBB2 gene, or both.

6. The method of claim 2, wherein the at least one genetic alteration is a T790M mutation in the EGFR gene.

7. The method of claim 2, wherein the second level of the at least one genetic alteration of ctDNA is at least about 90% lower than the first level of the at least one genetic alteration of ctDNA.

8. The method of claim 1, wherein:
detecting the first cfTL comprises detecting a first level of aneuploidy in the biological sample isolated from the subject at the first time point, wherein the first cfTL corresponds to the first level of aneuploidy; and
detecting the second cfTL comprises detecting a second level of aneuploidy in the biological sample isolated from the subject at the second time point, wherein the second cfTL corresponds to the second level of aneuploidy.

9. The method of claim 8, wherein detecting the first level of aneuploidy, detecting the second level of aneuploidy, or both comprises:
performing digital karyotyping, next generation sequencing, array-based methods, and combinations thereof.

10. The method of claim 8, comprising:
a) extracting a first sample of cell-free DNA from blood at the first time point;
ligating a low complexity pool of dual index barcode adapters to the first cell-free DNA sample to generate a first plurality of barcode adapter-ligated cell-free DNA segments;
capturing the first plurality of barcode adapter-ligated cell-free DNA segments;
eluting the non-captured cell-free DNA to generate a first non-captured cell-free DNA sample;
detecting the first level of aneuploidy in the first non-captured non-capture cell-free DNA; and
b) extracting a second sample of cell-free DNA from blood at the second time point;
ligating a low complexity pool of dual index barcode adapters to the cell-free DNA to generate a plurality of barcode adapter-ligated cell-free DNA segments;
capturing the plurality of barcode adapter-ligated cell-free DNA segments;
eluting the non-captured cell-free DNA to generate a second non-captured cell-free DNA sample;
detecting the second level of aneuploidy in the second non-captured non-capture cell-free DNA.

11. The method of claim 8, wherein a second level of at least one genetic alteration in circulating tumor DNA (ctDNA) in the biological sample isolated from the subject at the first time point is not substantially different than a first level of the at least one genetic alteration in circulating tumor DNA (ctDNA) in the biological sample isolated from the subject at the first time point.

12. The method of claim 1, wherein the biological sample obtained from the subject at the first time point, the second time point, or both comprises blood, plasma, serum, urine, cerebrospinal fluid, saliva, sputum, broncho-alveolar lavage, bile, lymphatic fluid, cyst fluid, stool, uterine lavage, vaginal fluids, ascites, and combinations thereof.

13. The method of claim 1, wherein the targeted therapy is a kinase inhibitor.

14-15. (canceled)

16. The method of claim 1, wherein the subject has been previously administered a different treatment or targeted therapy and the different treatment or targeted therapy was determined not to be therapeutically effective.

17. (canceled)

18. The method of claim 17, further comprising administering a therapeutic intervention to the subject.

19. (canceled)

20. The method of claim 1, wherein the cancer is selected from the group consisting of: a head and neck cancer, a central nervous system cancer, a lung cancer, a mesothelioma, an esophageal cancer, a gastric cancer, a gall bladder cancer, a liver cancer, a pancreatic cancer, a melanoma, an ovarian cancer, a small intestine cancer, a colorectal cancer, a breast cancer, a sarcoma, a kidney cancer, a bladder cancer, a uterine cancer, a cervical cancer, and a prostate cancer.

21. The method of claim 20, wherein the cancer is a lung cancer, and the lung cancer is non-small cell lung cancer.

22. The method of claim 20, wherein the cancer comprises a population of cancer cells that harbor an EGFR mutation, a ERBB2 mutation, or both.

23. The method of claim 1, the second time point is between about 1 week to about 4 weeks after the first time point.

24-25. (canceled)

26. A method of determining response to a targeted therapy in a subject having cancer, comprising:
detecting a first level of at least one genetic alteration in circulating tumor DNA (ctDNA) in a biological sample isolated from the subject at a first time point;
detecting a second level of the at least one genetic alteration in circulating tumor DNA (ctDNA) in a biological sample obtained from the subject at a second time point, wherein the subject has received at least one dose of the targeted therapy between the first time point and the second time point; and
identifying the subject as responding to the targeted therapy when the second level of the at least one genetic alteration is substantially increased as compared to the first level of the at least one genetic alteration.

27-28. (canceled)

29. A method of determining poor efficacy of a targeted therapy in a subject having cancer, comprising:
detecting a first cell-free tumor load (cfTL) in a biological sample isolated from the subject at a first time point;
detecting a second cfTL in a biological sample obtained from the subject at a second time point, wherein the subject has received at least one dose of the targeted therapy between the first time point and the second time point; and
identifying the targeted therapy as having poor efficacy in the subject when the subject exhibits a second cfTL that is not substantially reduced as compared to the first cfTL.

30-34. (canceled)
